SEQUENCE LISTING (3 sequences)

SEQ ID NO: 1 - DNA, 21 bp, Artificial Sequence
  Synthetic
tattacagcactagcagatca

SEQ ID NO: 2 - DNA, 39 bp, Artificial Sequence
  Synthetic
cttaagcttgccaccatgaagctctccctggtggccgcg

SEQ ID NO: 3 - DNA, 34 bp, Artificial Sequence
  Synthetic
aggcctcgagctacaactcatctttttctgctgt

(19) United States
(12) Patent Application Publication
(10) Pub. No.: US 2008/0039383 A1
(43) Pub. Date: Feb. 14, 2008

(54) Methods and compositions for inhibiting ER-stress induced cholesterol/triglyceride accumulation

(75) Inventors: Richard C. Austin, Ancaster (CA); Geoff Werstuck, Ancaster (CA)

Correspondence Address: MERCHANT & GOULD PC, P.O. BOX 2903, MINNEAPOLIS, MN, 55402-0903, US

(73) Assignee: HAMILTON CIVIC HOSPITAL RESEARCH DEVELOPMENT INC., Hamilton (CA)

(21) Appl. No.: 11/498,968

(22) Filed: Aug. 2, 2006

Related U.S. Application Data
(63) Continuation of application No. 10/130,592, filed on Sep. 20, 2002, now abandoned.
(60) Provisional application No. 60/166,114, filed on Nov. 16, 1999.

(51) Int. Cl. A61K 38/17

(52) U.S. Cl. 514/12

(57) ABSTRACT

The present invention provides methods for preventing the accumulation of cholesterol/triglycerides within mammalian cells. The present methods are based upon the surprising discovery that ER stress in a cell leads to cholesterol/triglyceride accumulation within the cell, which cholesterol/triglyceride accumulation is often a causative factor in the development of any of a number of conditions or diseases, such as atherosclerosis. The ER stress can be the result of any of a variety of causes, including homocysteine, viral infection, and hypoxia. Accordingly, counteracting the progression or the severity of ER stress can be used to inhibit the accumulation of cholesterol/triglycerides in said cell, thereby preventing or lessening the severity of any of a number of cholesterol-related diseases or conditions, e.g., atherosclerosis. In addition, the presence of ER stress in a cell can be used to diagnose a cholesterol associated disease, or to predict the propensity of a mammal to develop a disease.

### FIELD OF THE INVENTION

The invention relates to methods and compositions for modulating endoplasmic reticulum stress (“ER-stress”) induced cholesterol and/or triglyceride accumulation in cells.

### BACKGROUND OF THE INVENTION

It is estimated that close to 40 million adults in the United States have levels of blood cholesterol of 240 mg/dL or above. High levels of cholesterol in such a large part of the population has a major impact on public health, as such levels have been associated with various types of cardiovascular disease, including atherosclerosis, angina, heart disease, high blood pressure, stroke and other circulatory ailments. Such cardiovascular diseases are a major cause of mortality and morbidity in the United States (Ross (1993) Nature 362:801-809), claiming close to 1 million lives per year. In addition, obesity, diabetes, and male impotence may be associated with high cholesterol levels. Clearly, new methods for the detection, treatment, and prevention of high cholesterol levels and their associated diseases are needed.

The development of atherosclerosis is a complex, chronic process which is initiated at sites of endothelial cell (EC) injury, and which involves a series of cellular events and interactions that culminate in the formation of atherosclerotic lesions. These lesions are characterized by infiltration of monocytic cells into the subendothelium, smooth muscle cell proliferation and migration, cholesterol deposition, and elaboration of extracellular matrix (Ross (1993) Nature 362:801-809; Spady (1999) Circulation 100:576-578; Berliner et al. (1995) Circulation 91:2488-2496; Navab, et al. (1996) Arterioscler. Thromb. Vasc. Biol. 16, 831-842). Cholesterol-laden smooth muscle cells and macrophages, morphologically recognized as foam cells, are observed at all stages of lesion development and are key components of the atherosclerotic plaque. Traditionally, cholesterol and its oxidized derivatives are thought to accumulate in atherosclerotic lesions when cholesterol influx exceeds efflux. This would explain atherosclerosis in patients with lipid disorders.

Patients with hyperhomocysteinemia (HH) frequently develop atherosclerosis, but usually have normal serum lipid profiles (McCully (1996) Nat. Med. 2:386-389; Ueland and Refsum (1989) J. Lab. Clin. Med. 114:473-501; Clarke, et al., (1991) New Engl. J. Med. 324:1149-1155; Selhub, et al. (1995) New Engl. J. Med. 332, 286-291; Welch and Loscalzo (1998) New Engl. J. Med. 338:1042-1050; den heijer, et al. (1996) New Engl. J. Med. 334:759-762; Wilken and Dudman (1992), Lusis, Rotter, and Sparkes, (eds). Monogr. Hum. Genet. Basel, Karger, vol. 14, pp 311-324; Harker, et al (1974) N. Engl. J. Med. 291:537-543). In addition, as many as 401% of patients diagnosed with premature coronary artery disease, peripheral vascular disease or recurrent venous thrombosis are reported to have HH (McCully (1996) Nat. Med. 2:386-389; Ueland and Refsum (1989) J. Lab. Clin. Med. 114:473-501; Clarke, et al., (1991) New Engl. J. Med. 324:1149-1155; Selhub, et al. (1995) New Engl. J. Med. 332, 286-291; Welch and Loscalo (1998) New Engl. J. Med. 338:1042-1050; den heijer, et al (1996) New Engl. J. Med. 334:759-762). Although severe HH is not common, mild HH, which leads to premature atherosclerosis and thrombotic disease, occurs in approximately 5-7% of the general population (McCully (1996) Nat. Med. 2:386-389; Ueland and Refsum (1989) J. Lab. Clin. Med. 114:473-501; Welch and Loscalzo (1998) New Engl. J. Med. 338:1042-1050).

Homocysteine is a thiol-containing amino acid formed during the metabolism of methionine to cysteine. Once synthesized, homocysteine may be either metabolized to cysteine by the transsulfuration pathway or remethylated to methionine (McCully (1996) Nat. Med. 2:386-389; Ueland and Refsum (1989) J. Lab. Clin. Med. 114:473-501; Clarke, et al., (1991) New Engl. J. Med. 324:1149-1155; Selhub, et al. (1995) New Engl. J. Med. 332, 286-291; Welch and Loscalzo (1998) New Engl. J. Med. 338:1042-1050; den heijer, et al., (1996) New Engl. J. Med. 334:759-462; Wilken and Dudman (1992), Lusis, Rotter, and Sparkes, (eds). Monogr. Hum. Genet. Basel, Karger, vol. 14, pp 311-324). Deficiencies of any of the enzymes or cofactors necessary for the metabolism of homocysteine can result in dysfunctional intracellular homocysteine metabolism, thereby leading to HH.

A variety of independent reports now demonstrate a potential link between homocysteine and lipid metabolism. Histological examination of CBS-deficient mice having HH show liver hypertrophy with hepatocytes that are enlarged, multinucleated and filled with microvesicular lipid droplets (Watanabe et al. (1995) PNAS USA 92: 1585-1589), a finding consistent with that observed for virtually all human patients with homocystinuria (Mudd et al., (1989) in The Metabolic Basis of Inherited Disease, Scriver et al., eds., McGraw-Hill, New York, 6th Edition, pp 693-734). Furthermore, homocysteine induces the production and secretion of cholesterol in the human hepatoma cell line, HepG2 (O et al., (1998) Biochim. Biophys. Acta 1393:317-324). Homocysteine and cholesterol also act synergistically to further raise plasma homocysteine, cholesterol and triglyceride levels (Zulli et al., (1998) Life Sci. 62: 2192-2194). It has recently been shown in cultured vascular endothelial cells that homocysteine increases expression of the sterol regulatory element-binding protein-1 (SREBP-1), an ER membrane-bound transcription factor which functions to activate genes encoding enzymes in the cholesterol and triglyceride biosynthetic pathways. (Outinen et al., (1999) Blood 94: 959-967; Outinen et al., (1998) Biochem. J. 332:213-221). Despite these studies, the underlying mechanisms by which homocysteine leads to the development and progression of arthersclerosis are not understood.

ER stress is a broad term used to refer to various conditions that can interfere with the workings of the endoplasmic reticulum (for review, see, Pahl (1999) Physiolog. Rev. 79:683-701). For example, an accumulation of un- or misfolded proteins in the ER, glucose starvation, leading to protein accumulation in the ER, starvation of cholesterol, or any of a number of drugs or other agents that disturb ER function can cause ER stress. In response to ER step, cells initiate the production of a number of gene products, largely through new transcription, that counteract the causes of the ER stress. Depending on the cause of the stress, such initiated proteins can include those involved in protein folding, such as chaperone proteins, and other transcription factors, such as nuclear factor kappa B (NFκB) transcription factors (Pahl H L, Baeuerle P A, EMBO J. 1995 Jun. 1; 14(11):2580-8).

### SUMMARY OF THE INVENTION

It has now been discovered that ER stress, e.g., caused by elevated levels of homocysteine, plays a major, causative role in the accumulation of cholesterol and triglycerides in cells, and that this accumulation is associated with the development of any of a number of diseases and conditions, including cholesterol-associated diseases such as atherosclerosis and hepatic steatosis associated with hyperhomocysteinemia.

The present invention provides novel methods for the diagnosis, treatment, and prevention of numerous disorders and conditions associated with elevated cholesterol/triglyceride accumulation in cells. This invention is based on the surprising discovery that endoplasmic reticulum (ER) stress is a causative factor in the accumulation of cholesterol and triglycerides in cells. In particular, this ER stress, which is often the result of elevated levels of homocysteine, leads to an increase in cholesterol biosynthesis and/or cholesterol uptake by the cell experiencing the stress, thereby leading to the accumulation of cholesterol in the cell. This increase in intracellular cholesterol levels can lead to any of a number of diseases or conditions, including atherosclerosis and hepatic steatosis in hyperhomocysteinemia.

Broadly stated the present invention relates to a method of modulating cholesterol and/or triglyceride accumulation in a cell of a mammal comprising modifying an ER stress response or ER stress in the cell. “Modulate” or modulating” refers to a change or an alteration in the amount of intracellular cholesterol and/or triglycerides. Modulation may be an increase or a decrease in concentration, a change in characteristics, or any other change in the biological, functional, or other properties of cholesterol and/or triglycerides in the cell. “Modifying” refers to increasing or decreasing the severity of, or prolonging or shortening the duration of ER stress or an ER stress response in a cell. In an embodiment, the severity or duration of ER stress or an ER stress response is reduced or inhibited. The severity or duration of an ER stress response or ER stress may be reduced or inhibited by increasing the amount of, or inducing the activity or expression of an ER resident chaperone protein; increasing the amount of, or inducing a transcription factor (e.g. a Growth Arrest and DNA Damage transcription factor, or a cAMP Response Element Binding (CREB) transcription factor), or reducing or down-regulating the expression or activity of the low density lipoprotein (“LDL”) receptor. The severity or duration of an ER stress response may also be reduced or inhibited by inhibiting the expression or activity of, or reducing the amount of, a sterol regulatory element binding protein (e.& SREBP-1 or SREBP-2).

In one aspect, the present invention provides a method of inhibiting the accumulation of cholesterol in a cell of a mammal, the method comprising inhibiting an ER stress response or ER stress in the cell.

ER stress or an ER stress response may be induced by an agent or condition that adversely affects the function of the endoplasmic reticulum. In one embodiment, ER stress or an ER stress response is induced by homocysteine. In another embodiment, the mammal has a cholesterol-associated disease or condition (e.g. artherosclerosis, diabetes, hypertension, hyperhomocysteinemia). In another embodiment, ER stress or an ER stress response is induced by a viral infection. In another embodiment, ER stress or an ER stress response is induced by hypoxia. In another embodiment, the accumulation of cholesterol is a result of an increased level of cholesterol biosynthesis in the cell. In another embodiment, the accumulation of cholesterol is a result of an increased level of cholesterol uptake into the cell.

In another embodiment, the cell is an endothelial cell. In another embodiment, the cell is a smooth muscle cell. In another embodiment, the cell is a macrophage. In another embodiment, the cell is a hepatic cell. In another embodiment, the cell is present at an atherosclerotic lesion within the mammal.

An ER stress response or ER stress may be inhibited by modulating the expression or activity of an ER stress response gene or gene product (i.e. a gene or gene product associated with ER stress or an ER stress response, in particular, a gene or gene product that is expressed, produced, up-regulated, or down regulated in response to ER stress). In an embodiment, an ER stress response or ER stress is inhibited by increasing the amount of, or inducing the expression or activity of an ER resident chaperone protein in the cell. In another embodiment, the ER resident chaperone protein is a member of the group stress family, in particular GRP78/BiP. In another embodiment, the ER resident chaperone protein is GRP94, GRP72, Calreticulin, Calnexin, Protein disulfide isomeruse, cis/trans-Prolyl isomerase, or HSP47. In another embodiment, an ER stress response is inhibited by inhibiting the expression or activity of, or reducing the amount of a SREBP (e.g. SREBP-1 or SREBP-2) in the cell. In a further embodiment, an ER stress response or ER stress is inhibited by increasing the amount of, or inducing a transcription factor including a Growth Arrest and DNA Damage transcription factor, or a cAMP Response Element Binding (CREB) transcription factor. In a still further embodiment, an ER stress response or ER stress is inhibited by reducing or downregulating the expression or activity of the low density lipoprotein (“LDL”) receptor.

In a particular embodiment, ER stress or an ER stress response is inhibiting by administering a cytokine that induces expression of an ER resident chaperone protein, preferably IL-3.

In another aspect, the present invention provides a method of inhibiting a cholesterol-associated disease or condition, in particular atherosclerosis, in a mammal, the method comprising inhibiting ER stress or an ER stress response within a population of cells of the mammal, whereby the accumulation of cholesterol and/or triglycerides in the population of cells is inhibited.

In one embodiment, the atherosclerosis in the mammal is induced by homocysteine. In another embodiment, the mammal has hyperhomocysteinemia. In another embodiment, the population of cells comprises endothelial cells. In another embodiment, the population of cells comprises smooth muscle cells. In another embodiment, the population of cells comprises macrophages. In another embodiment, the population of cells comprises hepatic cells. In another embodiment, the population of cells is present at an atherosclerotic lesion within the mammal. In another embodiment, the ER stress response is inhibited by increasing the amount of, or inducing the expression or activity of an ER resident chaperone protein in the population of cells. In another embodiment, the ER resident chaperone protein is GRP78/BiP. In another embodiment, the ER resident chaperone protein is GRP94, GRP72, Calreticulin, Calnexin, Protein disulfide isomerase, cis/trans-Prolyl isomerase, or HSP47. In another embodiment, the ER stress response is inhibited by inhibiting the expression or activity of, or reducing the amount of a SREBP in the population of cells. In a further embodiment, an ER stress response or ER stress is inhibited by increasing the amount of, or inducing a transcription factor including a Growth Arrest and DNA Damage transcription factor, or a cAMP Response Element Binding (CREB) transcription factor. In a still further embodiment, an ER stress response or ER stress is inhibited by reducing or down regulating the expression or activity of the low density lipoprotein (“LDL”) receptor.

The invention contemplates the use of a modulator of ER stress or an ER stress response in the manufacture of a medicament for prevention or treatment of a cholesterol-associated disease or condition.

The invention also contemplates a pharmaceutical composition for the prevention or treatment of a cholesterol-associated disease or condition in a subject comprising a substance that induces the expression of an ER resident chaperone protein, said substance administered in a form and amount effective to reduce cholesterol and/or triglyceride accumulation in cells of the subject. In an embodiment, the substance is a cytokine, preferably IL-3.

In another aspect, the present invention provides a method of determining the propensity of a mammal to develop a cholesterol-associated disease or condition, the method comprising detecting the level of ER stress in a population of cells of the mammal.

In one embodiment, the cholesterol associated disease or condition is atherosclerosis. In another embodiment, the ER stress is detected by detecting the level or activity of a gene or gene product associated with ER stress. The gene or gene product may be GRP78, GADD153, GADD45, GADD34, ATF3, ATF4, ATF6, SREBP, GRP94, a NFκB transcription factor, LDL receptor, and/or YY1 (Yin Yang 1, GenBank NM 003403). In another embodiment, the population of cells comprises endothelial cells. In another embodiment, the population of cells comprises smooth muscle cells. In another embodiment, the population of cells comprises macrophages. In another embodiment, the population of cells comprises hepatic cells. In another embodiment, the population of cells is derived from an atherosclerotic lesion within the mammal.

The invention also provides a method for identifying a compound useful in the treatment or prevention of a cholesterol associated disease or condition comprising identifying a compound that inhibits ER stress or an ER stress response.

These and other aspects, features, and advantages of the present invention should be apparent to those skilled in the art from the following drawings and detailed description.

### DESCRIPTION OF THE DRAWINGS

The invention will be better understood with reference to the drawings in which:

FIG. 1 shows that homocysteine induces the steady-state mRNA levels of sterol regulatory element binding protein (SREBP), HMG-CoA reductase (HMG-CoA) and farnesyl diphosphate (FPP) synthase in HepG2 Cells. Equivalent amounts of total RNA (10 μg/lane) isolated from HepG2 cells cultured for 0, 2, 4, 8, or 18 hours in the presence of 5 mM homocysteine were examined for SREBP, HMG-CoA and FPP synthase mRNA induction by Northern blot analysis. Results demonstrate that homocysteine increased steady-state mRNA levels for all transcripts. As a positive control, cells were cultured for 18 hours in the presence of mevastatin (10 μg/ml), an HMG-CoA reductase inhibitor.

FIG. 2 demonstrates that homocysteine induces the expression of IPPI in HUVEC, HepG2 and human aortic smooth muscle cells (HASMC). Equivalent amounts of total RNA (10 μg/lane) isolated from HUVEC, HepG2 or HASMC cultured for 0, 2, 4, 8 or 18 hours in the presence of 5 mM homocysteine were examined by Northern blot analysis using an IPPI cDNA probe. Results demonstrate that homocysteine significantly increases IPPI mRNA levels in all cell lines. As a positive control for IPPI induction, cells were cultured for 18 hours in the presence of mevastatin (10 μg/ml), an HMG-CoA reductase inhibitor.

FIG. 3 shows the effect of various agents/conditions on steady-state mRNA levels of IPPI in HUVEC. In the upper panel, equivalent amounts of total RNA (10 μg/lane) isolated from HUVEC cultured for 4 hours in the absence or presence of either 5 mM homocysteine, glycine, homoserine, methionine, cysteine or 2 mM dithiothreitol (DTT) were examined by Northern blot analysis using an IPPI cDNA probe. Results demonstrate that only homocysteine and DTT significantly increased IPPI mRNA levels. Similar findings were observed for HepG2 and HASMC (data not shown). As a positive control for IPPI induction, HUVEC were cultured in lipoprotein-deficient (Lp) media for periods up to 24 hours (lower panel).

FIG. 4 shows the effect of endoplasmic reticulum (ER) stress agents on steady-state mRNA levels of IPPI. Equivalent amounts of total RNA (10 μg/lane) isolated from HepG2 cells cultured from 4 hours in the absence or presence of either homocysteine (5 mM), dithiothreitol (DTT) (5 mM), β-mercaptoethanol (5 mM), tunicamycin (10 μg/ml), or the Ca²⁺ ionophore A23187 (10 μM) were examined by Northern blot analysis using an IPPI cDNA probe. Results demonstrate that all of the ER stress agents increase IPPI mRNA levels. Similar findings were observed for HUVEC and HASMC (data not shown).

FIG. 5 are graphs showing the effect of homocysteine on intracellular total cholesterol. HUVEC, HASMC and HepG2 cells were incubated for 48 hr in media containing 0 to 5 mM homocysteine. Cells were washed in PBS, harvested in 0.2 M NaOH and lipids extracted as described in the Examples. Total cholesterol was normalized for protein content and values were expressed as percentage versus cells treated in the absence of homocysteine. Results are shown as the mean ±S.E.M. from three separate experiments. *p<0.05: level of statistical significance between indicated values and corresponding controls treated with 0 mM homocysteine.

FIG. 6 provides an analysis of cholesterol synthesis and efflux in HepG2 cells. Cells were incubated at 37° C. in the absence or presence of [¹⁴C]acetate for 0, 2, 4, or 8 hours. Radiolabeled cholesterol was extracted from cell lysates or media and resolved by thin layer chromatography (TLC) on Silica Gel G plates in petroleum etherdiethyl etheracetic acid (60:40:1 v/v). TLC plates were dried and subjected to autoradiography for 24 hours. Following autoradiography, the positions of the recovery-derived cholesterol was visualized by staining in iodine vapour.

FIG. 7 shows LDL binding to HUVEC, HASMC and HepG2 cells pre-treated with homocysteine. Cells, pre-treated with 0 or 5 mM homocysteine for 8 hours, were washed and then incubated in media containing 10 μg/ml BODIPY FL LDL (Molecular Probes, Inc. Eugene, Oreg.) for 2 hours at 37° C. Bound LDL was detected by fluorescence microscopy (magnification×375). HUVEC binding to acetylated (Ac) LDL was similarly down-regulated by homocysteine (not shown). AcLDL binding to HASMC and HepG2 was not detected.

FIG. 8 shows that heterozygous CBS deficient mice exhibit tissue specific cholesterol accumulation. Lipids were extracted from tissues of heterozygous CBS deficient mice (CBS+/−) and age-matched, wild type control mice (CBS+/+). Total cholesterol and cholesterol ester concentrations were determined and normalized to the total protein content of each tissue. Significant increases in cholesterol concentration were found in brain, kidney and lung. Data are the means ±standard error from 6 separate measurements on tissues from 2 wild type and 2 heterozygous CBS-deficient mice.

FIG. 9 shows stable overexpression of human GRP78/BiP in ECV304 cells. Equivalent amounts of total protein lysates (30 μg/lane) from wild-type ECV304 cells (ECV304), or cells stably transfected with either the vector pcDNA3.1(+) (ECV304-pcDNA) or the vector containing the full-length human GRP78/BiP cDNA (ECV304GRP78c1 or c2) were separated by SDS-polyacrylamide gel electrophoresis under reducing conditions. Gels were either stained with Coomassie Blue (upper panel) or immunostained with an anti-KDEL mAb which recognizes both GRP78/BiP and GRP94 (lower panel). The migration positions of GRP78 and GRP94 are shown by the arrowhead.

FIG. 10 shows immunolocalization of endogenous and transfected GRP78BiP in ECV304 cells. Wild-type ECV304 cells (top panel) or cells stably transfected with GRP78/BiP cDNA (lower panel) plated onto gelatin-coated glass coverslips were fixed, permeabilized and incubated with an anti-GRP78/BiP mAb (Santa Cruz Biotechnology). Antibody localization was detected with a FITC-conjugated goat anti-mouse IgG. Magnification×1000.

FIG. 11 shows that homocysteine does not induce the steady-state mRNA levels of IPPI in ECV304 cells that overexpress GRP78/BiP. Equivalent amounts of total RNA (10 μg/lane) isolated from wild-type, vector-transfected (ECV304-pcDNA3.1) or GRP78/BiP overexpressing ECV304 (ECV304-GRP78) cells cultured for 0, 4, 8, or 18 hours in the presence of 5 mM homocysteine were examined for IPPI mRNA induction by Northern blot analysis.

FIG. 12 is a graph showing intracellular homocysteine levels in HepG2 cells. HepG2 cells were cultured in the presence of 1 or 5 mM homocysteine. After 0, 2, 4, 8 and 24 h, cells were washed and lysed by three freeze/thaw cycles. Total intracellular homocysteine was determined using the Abbott IMx System and normalized to total protein. Data are the means ±standard error of 3 separate experiments.

FIG. 13 are immunoblots showing that homocysteine induces the expression of the ER stress response genes GRP78/BiP, GRP94 and GADD153. A. Equivalent amounts of total RNA (10 μg/lane) isolated from HepG2 cells cultured for 4 h in the absence (control) or presence of either 5 mM homocysteine, cysteine, methionine, homoserine, glycine, 2.5 mM DTT, or 10 μg/ml tunicamycin were examined by Northern blot analysis for GRP78/BiP and GADD153 mRNA induction. Control for equivalent RNA loading was assessed using a GAPDH cDNA probe. B. Whole cell lysates (40 μg total protein/lane) from HepG2 cells treated with 5 mM homocysteine for 0-36 h were separated on a 10% SDS-polyacrylamide gel under reducing conditions and immunostained with an anti-KDEL mAb that recognizes both GRP94 and GRP78/BiP.

FIG. 14 are immunoblots showing that homocysteine induces the activation and expression of SREBP-1 in HepG2 cells. (A) HepG2 cells were cultured in the absence or presence of 5 mM homocysteine for 2, 4, 8 or 18 h. Whole cell lysates (40 μg total protein/lane) were separated on 10% SDS-polyacrylamide gels under reducing conditions and immunostained with a mAb that recognizes both the precursor (P) and mature (M) forms of SREBP-1. (B) Northern blot analysis of total RNA (10 μg/lane) isolated from HepG2 cells cultured in the presence of 5 mM homocysteine for 0, 2, 4, 8 or 18 h. Blots were probed with a radiolabelled SREBP-1 cDNA. Control for equivalent RNA loading was assessed using a GAPDH cDNA probe.

FIG. 15 is an immunoblot showing that homocysteine induces the steady-state mRNA levels of isopentyl diphosphate:dimethylallyl diphosphate (IPP) isomerase, HMG-CoA reductase, and FPP synthase in HepG2 cells. Equivalent amounts of total RNA (10 μg/lane) isolated from HepG2 cells cultured for 0, 2, 4, 8 or 18 h in the presence of 5 mM homocysteine were examined for HMG-CoA reductase, IPP isomerase and FPP synthase mRNA induction by Northern blot analysis. Control for equivalent RNA loading was assessed using a GAPDH cDNA probe.

FIG. 16 is an immunoblot showing the effect of endoplasmic reticulum (ER) stress agents on steady-state mRNA levels of IPP isomerase in HepG2 cells. Equivalent amounts of total RNA (10 μg/lane) isolated from HepG2 cells cultured for 4 h in the absence (control) or presence of homocysteine (5 mM), DTT (2.5 mM), β-mercaptoethanol (5 mM), tunicamycin (10 μg/ml), or the Ca²⁺ ionophore A23187 (10 μM) were examined by Northern blot analysis using an IPP isomerase cDNA probe. Control for equivalent RNA loading was assessed using a GAPDH cDNA probe.

FIG. 17 are photographs showing the effect of homocysteine on LDL uptake in HUVEC, HASMC and HepG2. Cells treated in the absence or presence of 5 mM homocysteine for 8 hr were washed with media and PBS followed by incubation for an additional 2 hr at 37° C. in media containing 10 μg/ml BODIPY FL LDL. After washing with PBS, cells were fixed and LDL binding/uptake was detected by fluorescence microscopy (×375).

FIG. 18 are photographs showing hepatic morphology of CBS+/− mice fed control diet (A) or high methionine/low folate diet (B) for 10-16 weeks. The hepatocytes from the mice fed high methionine/low folate diet are enlarged and multinucleated, and contain extensive microvesicular and macrovesicular lipid with no apparent fibrosis or necrosis. Haematoxalin & Eosin staining; (×300).

FIG. 19 is an immunoblot showing that the livers of mice having diet-induced hyperhomocysteinemia contain elevated levels of mRNAs encoding GADD153 and LDL receptor proteins. Three week old C57BL6/J mice were fed a control diet (C), a high methionine diet (H or a combination high methionine/low folate diet (HMLF). After 2 weeks the animals were sacrificed and tissues harvested. Total RNA (10 μg/lane) isolated from the livers of 2 animals from each group was examined by Northern blot analysis using a GADD153 cDNA probe or LDL receptor cDNA probe. Control for equivalent RNA loading was assessed using a GAPDH cDNA probe.

### DETAILED DESCRIPTION OF THE INVENTION AND PREFERRED EMBODIMENTS

### 1. Introduction

The present invention provides methods for preventing the accumulation of cholesterol within mammalian cells. The present methods are based upon the surprising discovery that ER stress is a causative factor in the accumulation of cholesterol within cells, and often leads to the development of any of a number of conditions or diseases, such as atherosclerosis. Accordingly, counteracting the progression or the severity of ER stress can be used to inhibit the accumulation of cholesterol in a cell, thereby preventing or lessening the severity of any of a number of cholesterol-related diseases or conditions such as atherosclerosis. Further, the presence of ER stress in a cell can be used to diagnose a cholesterol-associated disease, or to predict the propensity of a mammal to develop such a disease.

Without being bound by the following theory, it is believed that an ER stress response, e.g., induced by elevated levels of intracellular homocysteine, results in the up-regulation of factors involved in cholesterol biosynthesis or uptake, producing a subsequent increase in cholesterol accumulation within the cell. While under normal circumstances, an increase in endogenous cholesterol leads to the down-regulation of LDL receptors, in the presence of ER stress, the sterol response element binding protein (SREBP) enhances LDL receptor expression, thereby counteracting this feedback mechanism. This continuous absorption of the synthesized cholesterol can explain why, in the case of homocysteine-induced atherosclerosis, there is not an observed correlation between elevated levels of plasma homocysteine and cholesterol, as the cholesterol accumulation is primarily local. The localized increases in cholesterol concentration may accelerate the accumulation of lipid in macrophages and smooth muscle cells in atherosclerotic lesions, thus promoting foam cell formation and plaque development. In addition, the discovery that hepatic cells accumulate cholesterol in response to ER stress, e.g., caused by homocysteine, helps explain why patients with severe hyperhomocysteinemia have fatty livers.

In numerous embodiments of this invention, the progression or severity of ER stress, or of an ER stress response, is inhibited. Such inhibition can be accomplished in any of a number of ways. For example, ER stress can be inhibited by inducing the expression of an ER resident chaperone protein, such as GRP78/BiP, or by inhibiting the expression or activity of an effector of an ER stress response, such as SREBP, or a transcription factor such as GADD153, ATF6, ATF3 or ATF4. The expression or activity of such proteins can be modulated in any of a number of ways, including by introducing a polynucleotide into cells within the mammal that encodes the protein, or an inhibitor of the protein. Alternatively, the cells can be treated with small molecules that affect, erg, the activity and/or expression of the proteins. The ER stress can be the result of any of a variety of causes, including, but not limited to, homocysteine, viral infection, hypoxia, reperfusion, and misfolding of proteins.

The inhibition of ER stress can be used to prevent or treat any of a number of cholesterol-associated diseases or conditions. In a preferred embodiment, ER stress or an ER stress response is inhibited in order to prevent the progression of atherosclerosis. Also preferred is the treatment of cholesterol associated diseases, e.g., atherosclerosis, that are caused by increased levels of homocysteine, e.g., in a mammal with hyperhomocysteinemia.

Because of the herein-described causative role of ER stress in the development of atherosclerosis and other cholesterol-associated diseases and conditions, the presence of such diseases or conditions, or the propensity of a mammal to develop such diseases or conditions, can be determined by detecting the presence of ER stress in cells within the mammal.

The present methods can be used to diagnose, determine the prognosis for, or treat, any of a number of cholesterol-associated conditions. In preferred embodiments, the conditions include atherosclerosis, or an atherosclerosis-related disease or condition such as angina, heart disease, high blood pressure, stroke and other circulatory ailments, and cyclosporin-induced cardiovascular disease. The methods of the invention can also be used to treat, prevent, or detect conditions associated with elevated cholesterol levels such as obesity, diabetes, and male impotence. In addition, the methods can be used to treat, prevent, or detect conditions that are caused by any ER stress-inducing factors, including, but not limited to, homocysteine, viral infection, hypoxia, shear stress, ultraviolet radiation, misfolding of proteins, ER protein accumulation, or any drug or agent that causes ER stress as-described, for example, in Pahl (1999) Physiol. Rev. 79:683-701.

The diagnostic methods of this invention can be used in any mammal, including, but not limited to, humans and other primates, canines, felines, murines, bovines, equines, ovines, porcines, and lagomorphs.

Kits are also provided for carrying out the herein-disclosed diagnostic and therapeutic methods.

### II. Definitions

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1994); Animal Cell Culture R. I. Freshney, ed. (1986); Immobilized Cells and enzymes IRL Press, (1986); and B. Perbal, A Practical Guide to Molecular Cloning (1984).

“ER stress” or “endoplasmic reticulum stress” refers to any of a number of cellular conditions whereby the function of the endoplasmic reticulum is disturbed, thereby leading to a response from the cell (“ER stress response”). Included in “ER stress” conditions are UPR, or “unfolded protein response,” which occurs following an accumulation of un- or misfolded proteins in a cell. UPR leads to the activation of a signaling pathway and the ultimate production of chaperone proteins, such as BiP/GRP78 (see, e.g., Brewer et al. (1997) EMBO J. 16:7207-7216). Other causes of ER stress can include glucose starvation, protein accumulation, cholesterol starvation, and others. Each particular cause of ER stress can provoke a particular response, involving a particular suite of gene expression.

An “ER resident chaperone protein” refers to any protein, present in the ER, that acts to facilitate the folding, assembly, or translocation of proteins (see, e.g., Ellis et al., (1989) Trends Biochem Sci 14(8):339-42; Ruddon et al., (1997) J. Biol. Chem. 272:3125-3128). As used herein, “ER resident chaperone proteins” can refer to any protein that facilites protein folding, assembly, or translocation, and which is naturally present in the ER or which is modified to be present in the ER, for example by the recombinant addition of a signal sequence and/or other ER localization domains. Examples of ER resident chaperone proteins include, but are not limited to, BiP/GRP78, GRP94, GRP72, Calreticulin, Calnexin (08, IP90), TRAP or p28, c tas-Prolyl isomerase, Protein disulfide isomerase, and others (see, e.g., Ruddon et al., supra), or proteins that are substantially identical thereto.

“Transcription factor” herein means a factor that regulates the transcription of proteins associated with ER stress or an ER stress response. A transcription factor may be a Growth Arrest And DNA Damage (GADD) transcription factor, including but not limited to GADD153 (a.k.a. C/EBP homologous protein or CHOP), GADD45, and GADD34 (Outinen, P A et al, 1998, 1999; Wang, X. Z. et al Mol. Cell. Biol. 16, 4273-4280; Takekawa, M. and Saito, H., Cell 95 (4), 521-530 (1998); Hollander, M. C. et al, J. Biol. Chem. 272 (21), 13731-13737 (1997)). A transcription factor may also be a cAMP Response Element Binding (CREB) transcription factor, including but not limited to ATF-6, ATF-3, and ATF-4 (Haze, K, et al. 1999, Wang, Y., et al. 2000; Cai, Y et al Blood 96, 2140-2148; Karpinski, B. A. et al Proc Natl Acad Sci USA 1992 Jun. 1; 89(11):4820-4).

“Providing a biological sample” means to obtain a biological sample for use in the methods described in this invention. Most often, this will be done by removing a sample of cells from an animal, but can also be accomplished by using previously isolated cells (e.g., isolated by another person, at another time, and/or for another purpose), or by performing the methods of the invention in vivo.

A “control sample” refers to a sample of biological material representative of a healthy mammal without elevated levels of ER stress or cholesterol accumulation. This sample can be removed from an animal expressly for use in the methods described in this invention, or can be any biological material representative of healthy mammals. A control sample can also refer to an established level of ER stress, representative of mammals without elevated ER stress or cholesterol, that has been previously established based on measurements from healthy animals. If a detection method is used that only detects an ER stress-related polypeptide or polynucleotide when a level higher than that typical of a healthy mammal is present, i.e., an immunohistochemical assay giving a simple positive or negative result, this is considered to be assessing the level of the polypeptide or polynucleotide in comparison to the control level, as the control level is inherent in the assay.

A level of a polypeptide or polynucleotide that is “expected” in a control sample refers to a level that is representative of healthy mammals, and from which an elevated, or diagnostic, presence of a polypeptide or polynucleotide can be distinguished. Preferably, an “expected” level will be controlled for such factors as the age, sex, medical history, etc. of the mammal, as well as for the particular biological sample being tested.

An “increased” or “elevated” level of a polypeptide or polynucleotide refers to a level of the polynucleotide or polypeptide, that, in comparison with a control level, is detectably higher. The method of comparison can be statistical, using quantified values, or can be compared using nonstatistical means, such as by a visual, subjective assessment by a human.

As used herein, a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (e.g., 7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

When a quantified level of an ER stress or ER stress-response associated protein or polynucleotide falls outside of a given confidence interval for a normal level of the protein or polynucleotide, the difference between the two levels is said to be “statistically significant” If a test value falls outside of a given confidence interval for a normal level of the protein or polynucleotide, it is possible to calculate the probability that the test value is truly abnormal and does not simply represent a normal deviation from the average. In the present invention, a difference between a test sample and a control can be termed “statistically significant” when the probability of the test sample being a normal deviation from the average can be any of a number of values, including 0.15, 0.1, 0.05, and 0.01. Numerous sources teach how to assess statistical significance, such as Freund, J. E. (1988) Modern elementary statistics, Prentice-Hall.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window or designated region, as measured using one of the following sequence comparison algorithms or by manual alignment and visual inspection.

The phrase “substantially identical,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et alt eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to crate the alignment PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (http://www.ncbi.ntm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=4 and a comparison of both strands. For amino acid sequences, the BLASTP program uses as defaults a wordlength of 3, and expectation (E) of 10, and the BLOSUM62 scoring matrix (see, Henikoff & Henikoff, Proc. Natl. Acad Sci USA 89:10915 (1989)) alignments (B) of 50, expectation (E) of 10, M=5, N=4, and a comparison of both strands.

The BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent hybridization conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 83 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. Washes can be performed, e.g., for 2, 5, 10, 15, 30, 60, or more minutes.

Nucleic acids that do not hybridize to each other under stringent hybridization conditions are still substantially identical if the polypeptides that they encode are substantially identical. This occurs, for example, when a copy of a nucleic acid is created using the maximum codon degeneracy permitted by the genetic code. In such cased, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies may exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552-554)

For preparation of monoclonal or polyclonal antibodies, any technique known in the art can be used (see, e.g., Kohler & Milstein, (1975) Nature 256:495-497; Kozbor et al., (1983) Immunology Today 4: 72; Cole et al, (1985), pp. 77-96 in Monoclonal Antibodies ad Cancer Therapy, Alan R Liss, Inc.). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., (1990) Nature 348:552-554; Marks et al., (1992) Biotechnology 10:779-783).

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

Specific binding to an antibody under such conditions may require an antibody that is selected for its specificity for a particular protein. For example, polyclonal antibodies raised to a particular polypeptide can be selected to obtain only those polyclonal antibodies that are specifically immunoreactive with the polypeptide and not with other proteins, except for polymorphic variants, orthologs, and alleles of the polypeptide. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind to a protein, as defined above.

### III. Inhibiting ER Stress in Cells

In numerous embodiments of the present invention, ER stress is inhibited within one or more cells of a mammal. ER stress can be inhibited in any of a number of ways, including by increasing the expression or activity of a chaperone protein in the ER or by counteracting the effects of an ER stress response, and can be inhibited, for example, to prevent any of a number of cholesterol-associated conditions and diseases, including atherosclerosis, heart disease, angina, high blood pressure, stroke, and other cardiovascular conditions, diabetes, obesity, and male impotence.

The methods described herein can be used to inhibit ER stress, or an ER stress response, in any of a number of cells within a mammal. Preferably, the cells are restricted to the cells undergoing ER stress and accumulating cholesterol and/or triglycerides, for example endothelial or macrophage cells (including foam cells) at an atherosclerotic lesion.

Such ER stress can be the result of any of a number of causes, including, but not limited to, homocysteine (e.g., in a mammal with hyperhomocysteinemia), hypoxia, cholesterol starvation, glucose starvation, shear stress, protein misfolding, viral infection, or any drug or agent that interferes with ER function.

A. Expressing or Activating ER Resident Chaperone Proteins

In an embodiment of the invention, an ER resident chaperone protein is expressed or activated in a cell to protect the cell from ER stress, thereby preventing the accumulation of cholesterol in the cell. In a particularly preferred embodiment, the expression or activity of GRP78/BiP (see, e.g., Kozutsumi et al. (1989) J Cell Sci Suppl 11:115-37; Ting et al. (1988) DNA 7(4):275-86; GenBank Accession No. M19645) is increased. In addition to GRP78/BiP, any other ER resident chaperone protein, such as GRP94 (see, e.g., Sorger et al. (1987) J Mol Biol 194(2):341-4; see, e.g., GenBank Accession No. M26596), calnexin (see, e.g., Wada et al. (1991) J. Biol. Chem. 266, 19599-19610; GenBank Accession No. M94859), and calreticulin (see, e.g., Michalak et al. (1992) Biochem J285 (Pt 3):681-92; Fliegel et al. (1989) J Biol Chem 264(36):21522-8; GenBank Accession No. NM_{—}004343), can be used. It will be appreciated that any variant, derivative, fragment, or allele of any of these genes or gene products, or substantially identical genes or gene products, or indeed any factor that can inhibit, suppress, or prevent ER stress, can be used, and that the expression of the gene can be induced using any of a number of methods, including, but not limited to, introducing nucleic acids encoding the gene product into cells in vivo, or by administering to a mammal a compound that induces the expression of the gene.

The synthesis of an ER resident chaperone protein may be regulated i.e. activated, at the level of transcription. Thus, the level of a transcription factor that upregulates transcription of an ER resident chaperone protein may be increased or induced in a cell to prevent the accumulation of cholesterol and/or triglycerides in the cell.

In certain embodiments, a growth factor will be administered to the cell that induces the expression of ER chaperone proteins. For example, IL-3 and other cytokines have been shown to induce the expression of ER chaperones such as GRP78/BiP and GRP94. See, e.g., Brewer et al., (1997) EMBO J. 16:7207-7216.

1. Expressing Chaperone Proteins and Other ER-Stress Inhibitors in Cells

In numerous embodiments, one or more nucleic acids, e.g., a GRP78/BiP polynucleotide, will be introduced into cells, in vitro or in vivo. Accordingly, the present invention provides methods, reagents, vectors, and cells useful for the expression of GRP78/BiP and other ER resident chaperone proteins and nucleic acids using in vitro (cell-five), ex vivo or in vivo (cell or organism-based) recombinant expression systems.

For use in the present invention, any of the well known procedures for introducing foreign nucleotide sequences into host cells may be used. These include the use of calcium phosphate transfection, spheroplasts, electroporation, liposomes, microinjection, plasma vectors, viral vectors and any of the other well known methods for introducing cloned genomic DNA, cDNA, synthetic DNA or other foreign genetic material into a host cell (see, e.g., Berger and Kimmel, Guide to Molecular Cloning Techniques, Methods in Enzymology volume 152 Academic Press, Inc., San Diego, Calif. (Berger), F. M. Ausubel et al., eds., Current Protocols, a joint venture between Greene Publishing Associates, Inc. and John Wiley & Sons, Inc., (supplemented through 1999), and Sambrook et al., Molecular Cloning—A Laboratory Manual (2nd Ed.), Vol. 1-3, Cold Spring Harbor Laboratory, Cold Spring Harbor, N.Y., 1989.

Preparation of various polynucleotides and vectors useful in the present invention are well known. General texts which describe methods of making recombinant nucleic acids include Sambrook et al., supra; Ausubel et al., supra, and Berger and Kimmel, Guide to Molecular Cloning Techniques, Methods in Enzymology, volume 152 Academic Press, Inc., San Diego, Calif. (Berger). In numerous embodiments of this invention, nucleic acids will be inserted into vectors using standard molecular biological techniques. Vectors may be used at multiple stages of the practice of the invention, including for subcloning nucleic acids encoding, e.g., components of proteins or additional elements controlling protein expression, vector selectability, etc. Vectors may also be used to maintain or amplify the nucleic acids, for example by inserting the vector into prokaryotic or eukaryotic cells and growing the cells in culture.

Product information from manufacturers of biological reagents and experimental equipment also provide information useful in known biological methods such as cloning. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc, GIBCO BRL Life Technologies, Inc. (Gaithersberg, Md.), Fluka Chemica-Biochemika Analytika (Fluka Chemie AG, Bucds, Switzerland), Invitrogen, San Diego, Calif., Applied Biosystems Foster City, Calif.), Digene Diagnostics, Inc. (Beltsville, Md.) as well as many other commercial sources known to one of skill. These commercial suppliers produce extensive catalogues of compounds, products, kits, techniques and the like for performing a variety of standard methods.

A convenient method of introducing the polynucleotides into cells in vivo and in vitro involves the use of viral vectors, e.g., adenoviral vector mediated gene delivery (see, e.g., Chen et al. (1994) Proc. Nat'l. Acad. Sci. USA 91: 3054-3057; Tong et al. (1996) Gynecol. Oncol. 61: 175-179; Clayman et al. (1995) Cancer Res. 5: 1-6; O'Malley et al. (1995) Cancer Res. 55: 1080-1085; Hwang et al. (1995) Am. J. Respir. Cell Mol. Biol. 13: 7-16; Haddada et al. (1995) Curr. Top. Microbiol. Immunol. 199 (Pt, 3): 297-306; Addison et al. (1995) Proc. Nat'l. Acad. Sci. USA 92: 8522-8526; Colak et al. (1995) Brain Res. 691: 76-82; Crystal (1995) Science 270: 404-410; Elshami et al. (1996) Human Gene Ther. 7: 141-148; Vincent et al. (1996) J. Neurosurg. 85: 648-654); and retroviral vectors (see, e.g., Marx et al. Hum Gene Ther 1999 May 1; 10(7):1163-73; Mason et al., Gene Ther 1998 August; 5(8):1098-104). In addition, replication-defective retroviral vectors harboring a therapeutic polynucleotide sequence as part of the retroviral genome have also been used, particularly with regard to simple MuLV vectors. See, e.g., Miller et al. (1990) Mol. Cell. Biol. 10:4239 (1990); Kolberg (1992) J. NIH Res. 4:43, and Cornetta et al. Hum. Gene. Ther. 2:215 (1991)). Other suitable retroviral vectors include lentiviruses (Klimatcheva et al., (1999) Front Biosci 4:D481-96). Other viral vectors that can be used in the present invention include vectors derived from adeno-associated viruses (Bueler (1999) Biol Chem 380(6):613-22; Robbins and Chivizzani (1998) Pharmacol Ther 80(1):3547), herpes simplex viruses (Krisky et al., (1998) Gene Ther 5(11): 1517-30), and others.

Plasmid vectors can also be delivered as “naked” DNA or combined with various transfection-facilitating agents. Numerous studies have demonstrated the direct administration of naked DNA, e.g., plasmid DNA, to cells in vivo (see, e.g., Wolff, Neuromuscul Disord 1997 July; 7(5):314-8, Nomura et al., Gene Ther. 1999 January; 6(1):121-9). For certain applications it is possible to coat the DNA onto small particles and project genes into cells using a device known as a gene gun.

Plasmid DNA can also be combined with any of a number of transfection-facilitating agents. The most commonly used transfection facilitating agents for plasmid DNA in vivo have been charged and/or neutral lipids (Debs and Zhu (1993) WO 93/24640 and U.S. Pat. No. 5,641,662; Debs U.S. Pat. No. 5,756,353; Debs and Zhu Published EP Appl. No. 93903386; Mannino and Gould-Fogerite (1988) BioTechniques 6(7): 682-691; Rose U.S. Pat. No. 5,279,833; Brigham (1991) WO 91/06309 and U.S. Pat. No. 5,676,954; and Felgner et al. (1987) Proc. Natl. Acad. Sci. USA 84: 7413-7414). Additional useful liposome-mediated DNA transfer methods, other than the references noted above, are described in U.S. Pat. No. 5,049,386, U.S. Pat. No. 4,946,787; and U.S. Pat. No. 4,897,355; PCT publications WO 91/17424, WO 91/16024; Wang and Huang, 1987, Biochem. Biophys. Res. Commun. 147: 980; Wang and Huang, 1989, Biochemistry 28: 9508; Litzinger and Huang, 1992, Biochem. Biophys. Acta 1113:201; Gao and Huang, 1991, Biochem. Biophys. Res. Commun. 179: 280. Immunoliposomes have been described as carriers of exogenous polynucleotides (Wang and Huang, 1987, Proc. Natl. Acad. Sci. U.S.A. 84:7851; Trubetskoy et al 1992, Biochem. Biophys. Acta 1131:311) and may have improved cell type specificity as compared to liposomes by virtue of the inclusion of specific antibodies which presumably bind to surface antigens on specific cell types. Behr et al., 1989, Proc. Natl. Acad. Sci. U.S.A. 86:6982 report using lipopolyamine as a reagent to mediate transfection itself, without the necessity of any additional phospholipid to form liposomes.

Lipid carriers usually contain a cationic lipid and a neutral lipid. Most in vivo transfection protocols involve forming liposomes made up of a mixture of cationic and neutral lipid and complexing the mixture with a nucleic acid. The neutral lipid is often helpful in maintaining a stable lipid bilayer in liposomes used to make the nucleic acid:lipid complexes, and can significantly affect transfection efficiency. Liposomes may have a single lipid bilayer (unilamellar) or more than one bilayer (multilamellar). They are generally categorized according to size, where those having diameters up to about 50 to 80 nm are termed “small” and those greater than about 80 to 1000 nm, or larger, are termed “large.” Thus, liposomes are typically referred to as large unilamellar vesicles (LUVs), multilamellar vesicles (MLVs) or small unilamellar vesicles (SUVs).

Cationic liposomes are typically mixed with polyanionic compounds (including nucleic acids) for delivery to cells. Complexes form by charge interactions between the cationic lipid components and the negative charges of the polyanionic compounds.

A wide variety of liposomal formulations are known and commercially available and can be tested in the assays of the present invention for precipitation, DNA protection, pH effects and the like. Because liposomal formulations are widely available, no attempt will be made here to describe the synthesis of liposomes in general. Two references which describe a number of therapeutic formulations and methods are WO 96/40962 and WO 96/40963.

Cationic lipid-nucleic acid transfection complexes can be prepared in various formulations depending on the target cells to be transfected. While a range of lipid-nucleic acid complex formulations will be effective in cell transfection, optimal conditions are determined empirically in the desired system. Lipid carrier compositions are evaluated, e.g., by their ability to deliver a reporter gene (e.g., CAT, which encodes chloramphenicol acetyltransferase, luciferase, β-galactosidase, or GFP) in vitro, or in vivo to a given tissue type in an animal, or in assays which test stability, protection of nucleic acids, and the like.

The lipid mixtures are complexed with nucleic acids in different ratios depending on the target cell type, generally ranging from about 6:1 to 1:20 μg nucleic acid:nmole cationic lipid.

For mammalian host cells, viral-based and nonviral, e.g., plasmid-based, expression systems are provided. Nonviral vectors and systems include plasmids and episomal vectors, typically with an expression cassette for expressing a protein or RNA, and human artificial chromosomes (see, e.g., Harrington et al., 1997, Nat Genet. 15:345). For example, plasmids useful for expression of polynucleotides and polypeptides in mammalian (e.g., human) cells include pcDNA3.1/His, pEBVHis A, B & C, (Invitrogen, San Diego Calif.), MPSV vectors, others described in the Invitrogen 1997 Catalog (Invitrogen Inc, San Diego Calif.), which is incorporated in its entirety herein, and numerous others known in the art for other proteins.

Useful viral vectors include vectors based on retroviruses, adenoviruses, adeno-associated viruses, herpes viruses, vectors based on SV40, papilloma virus, HBP Epstein Barr virus, vaccinia virus vectors and Semlilki Forest virus (SFV). SFV and vaccinia vectors are discussed generally in Ausubel it al., supra, Ch. 16. These vectors are often made up of two components, a modified viral genome and a coat structure surrounding it (see generally, Smith, 1995, Ann. Rev. Microbiol. 49: 807), although sometimes viral vectors are introduced in naked form or coated with proteins other than viral proteins. However, the viral nucleic acid in a vector may be changed in many ways, for example, when designed for gene therapy. The goals of these changes are to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest.

Thus, viral vector nucleic acids generally comprise two components: essential cis-acting viral sequences for replication and packaging in a helper line and the transcription unit for the exogenous gene. Other viral functions are expressed in trans in a specific packaging or helper cell line. Adenoviral vectors (e.g., for use in human gene therapy) are described in, e.g., Rosenfeld et al., 1992, Cell 68: 143; PCT publications WO 94/12650; 94/12649; and 94/12629. In cases where an adenovirus is used as an expression vector, a sequence may be ligated into an adenovirus transcription/translation complex consisting of the late promoter and tripartite leader sequence. Insertion in a nonessential E1 or E3 region of the viral genome will result in a viable virus capable of expressing in infected host cells (Logan and Shenk, 1984, Proc. Natl. Acad Sci., 81:3655). Replication-defective retroviral vectors harboring a therapeutic polynucleotide sequence as part of the retroviral genome are described in, e.g., Miller et al., 1990, Mol. Cell. Biol. 10: 4239; Kolberg, 1992, J. NIH Res. 4: 43; and Cornetta et al., 1991, Hum. Gene Ther. 2: 215. In certain embodiments, the surface of the virus can be coated, e.g., by covalent attachment, with polyethylene glycol (PEG; see, e.g., O'Riordan et al., (1999) Hum Gene Ther. 10(8): 1349-58.). Such “PEGylation” of viruses can impart various benefits, including increasing the infectivity of the virus, and lowering the host immune response to the virus.

A variety of commercially or commonly available vectors and vector nucleic acids can be converted into a vector for use in the invention by cloning a polynucleotide (e.g. a polynucleotide encoding an ER resident chaperone protein) into the commercially or commonly available vector. A variety of common vectors suitable for this purpose are well known in the art. For cloning in bacteria, common vectors include pBR322-derived vectors such as pBLUESCRIPT™, and bacteriophage derived vectors. In yeast, vectors include Yeast Integrating plasmids (e.g., YIp5) and Yeast Replicating plasmids (the YRp series plasmids) and pGPD2. Expression in mammalian cells can be achieved using a variety of commonly available plasmids, including pSV2, pBC12B1, and p91023, as well as lytic virus vectors (e.g., vaccinia virus, adeno virus, and baculovirus), episomal virus vectors (e.g., bovine papillomavirus), and retroviral vectors (e.g., murine retroviruses).

Typically, a nucleic acid subsequence encoding a polypeptide, e.g., an ER resident chaperone protein, is placed under the control of a promoter. A nucleic acid is “operably linked” to a promoter when it is placed into a functional relationship with the promoter. For instance, a promoter or enhancer is operably linked to a coding sequence if it increases or otherwise regulates the transcription of the coding sequence. Similarly, a “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include promoters and, optionally, introns, polyadenylation signals, and transcription termination signals. Additional actors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

An extremely wide variety of promoters are well known, and can be used in the vectors of the invention, depending on the particular application. Ordinarily, the promoter selected depends upon the cell in which the promoter is to be active. In mammalian cell systems, promoters from mammalian genes or from mammalian viruses are often appropriate. Suitable promoters may be constitutive, cell type-specific, stage-specific, and/or inducible or repressible (e.g., by hormones such as glucocorticoids). Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Other expression control sequences such as ribosome binding sites, transcription termination sites and the like are also optionally included. For E. coli, example control sequences include the 17, trp, or lambda promoters, a ribosome binding site and preferably a transcription termination signal. For eukaryotic cells, the control sequences typically include a promoter which optionally includes an enhancer derived from immunoglobulin genes, SV40, cytomegalovirus, a retrovirus (e.g., an LTR based promoter) etc., and a polyadenylation sequence, and may include splice donor and acceptor sequences.

B. Inhibiting ER Stress Response

In numerous embodiments, cholesterol accumulation is inhibited in a cell by inhibiting the expression or activity of a gene associated with an ER stress response. For example, ER stress has been discovered to cause the expression of sterol regulatory element binding protein (SREBP), which in turn induces the expression of a number of genes involved in cholesterol biosynthesis and uptake, such as isopentyl diphosphate:dimethylallyl diphosphate isomerase (IPPI), 3-hydroxy-3-methylglutaryl coenzyme A (HMG CoA) reductase, and farnesyl diphosphate (FPP) synthase, as well as LDL receptors. The expression or activity of any of these well known genes or gene products (see, e.g., Outinen et al., (1999) Blood 94:959-967) can be inhibited in any of a number of ways, e.g., by decreasing the level of mRNA or protein in a cell using, e.g., ribozymes or antisense compounds, or by introducing an inhibitor of a protein using, e.g., antibodies, small molecule inhibitors, dominant negative forms of the proteins, etc. Preferably, the level of the protein or protein activity is lowered to a level typical of a cell in the absence of ER stress but the level may be reduced to any level that is sufficient to decrease the accumulation of cholesterol in the cell, including to levels above or below those typical of cells without ER stress.

In certain embodiments, the level of expression of an ER stress induced gene is downregulated, or entirely inhibited, by the use of antisense polynucleotide, i.e., a nucleic acid complementary to, and which can preferably hybridize specifically to, a coding mRNA nucleic acid sequence, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally occurring subunits or their close homologs. Antisense polynucleotides may also have altered sugar moieties or inter-sugar linkages. Exemplary among these are the phosphorothioate and other sulfur containing species which are known for use in the art All such analogs are comprehended by this invention so long as they function effectively to hybridize with an mRNA.

Such antisense polynucleotides can be readily synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

In addition to antisense polynucleotides, ribozymes can be used to target and inhibit transcription of an ER stress response gene. A ribozyme is an RNA molecule that catalytically cleaves other RNA molecules. Different kinds of ribozymes have been described, including group I ribozymes, hammerhead ribozymes, hairpin ribozymes, RNAse P, and axhead ribozymes (see, e.g., Castanotto et al. (1994) Adv. in Pharmacology 25: 289-317 for a general review of the properties of different ribozymes).

The general features of hairpin ribozymes are described, e.g., in Hampel et al (1990) Nucl. Acids Res. 18: 299-304; Hampel et al. (1990) European Patent Publication No. 0 360 257; U.S. Pat. No. 5,254,678. Methods of preparing are well known to those of skill in the art (see, e.g., Wong-Staal et al., WO 94/26877; Ojwang et al. (1993) Proc. Natl. Acad. Sci. USA 90: 6340-6344; Yamada et al. (1994) Human Gene Therapy 1: 3945; Leavitt et al. (1995) Proc. Natl. Acad. Sci. USA 92: 699-703; Leavitt et al. (1994) Human Gene Therapy 5: 1151-120; and Yamada et al. (1994) Virology 205: 121-126).

The activity of an ER stress response protein can also be decreased using an inhibitor of the protein. This can be accomplished in any of a number of ways, including by providing a dominant negative polypeptide, e.g., a form of the protein that itself has no activity and which, when present in the same cell as a functional protein, reduces or eliminates the activity of the functional protein (see, e.g., Herskowitz (1987) Nature 329(6136):219-22). Also, inactive polypeptide variants (muteins) can be used, e.g., by screening for the ability to inhibit protein activity. Methods of making muteins are well known to those of skill (see, e.g., U.S. Pat. Nos. 5,486,463, 5,422,260, 5,116,943, 4,752,585, 4,518,504). In addition, any small molecule, e.g., any peptide, amino acid, nucleotide, lipid, carbohydrate, or any other organic or inorganic molecule can be screened for the ability to bind to or inhibit protein activity, e.g. using high throughput screening methods as taught above, and screening for a loss of any measure of the level or activity of an ER stress response gene or gene product. For example, a decrease in the RNA or protein level in cells can be detected using standard methods following administration of a test compound, as can a decrease in protein activity by detecting, e.g., the amount of target gene expression for ER stress response proteins that are transcription factors or signaling molecules that indirectly cause gene expression.

C. Screening for Inhibitors of ER Stress

In an embodiment, the present invention provides methods for identifying compounds useful in the treatment or prevention of cholesterol-associated diseases, e.g., atherosclerosis, the method comprising identifying a compound that inhibits ER stress, as described herein. Such inhibitors can act, e.g., by inducing the expression or activity of a gene or gene product that itself inhibits ER stress, such as an ER resident chaperone protein such as GRP78/BiP, or by inhibiting the expression or activity of an ER stress response protein such as SREBP. For example, to identify agents that induce the expression of an ER resident chaperone, e.g., GRP78/BiP, a preferred “screening” method involves (i) contacting a cell capable of expressing GRP78/BiP with a test agent, and (ii) detecting the level of GRP78/BiP expression (e.g. as described above), where an increased level of expression as compared to the level of expression in a cell not contacted with the test agent indicates that the test agent increases or induces the expression of the protein. Such modulators of expression or activity of an ER stress or ER stress response related protein can also involve detecting the ability of a test agent to bind to or otherwise interact with the protein of interest, or of a nucleic acid sequence, e.g., a promoter, encoding or regulating the expression of the protein. In addition, any agent that inhibits ER stress, independent of its effect on the herein-described genes and gene products, can be screened for the ability to inhibit ER stress. The ability of such test agents, or indeed of any of the herein-described genes, gene products, or any derivative, variant, fragment, or allele thereof, to inhibit or otherwise counteract ER stress can be tested using any of a number of means. For example, the induction of ER stress can be detected by detecting the expression or activation of any ER stress response gene or gene product, including, but not limited to, GRP78/BiP, a NFκB transcription factor, GADD153, GADD45, ATF-6, ATF-3, Id-1, ATF4, YY1, LDL receptor, cyclin Di, FRA-2, glutathione peroxidase, NKEF-B PAG, superoxide dismutase, and clusterin (Outinen et al. (1999) Blood 94:959-967; Outinen et al. (1998) Biochem. J. 332:213-221). In addition, ER stress-inducing ability can be detected using a “cell-killing” type assay, where the ability of an agent to kill a cell by ER stress can be determined by comparing the ability of the agent to kill cells in normal cells or in cells expressing an ER protecting factor, such as GRP78/BiP. Agents that kill cells only in the absence of such protective factors are identified as ER stress-inducing factors. See, e.g., Morris et al. (1997) J. Biol. Chem. 272:4327-34). Agents that affect the level of misfolded proteins can also be used, e.g., to detect modulation of ER stress, by, e.g., detecting misfolded proteins by virtue of their ability to bind to GRP78/BiP.

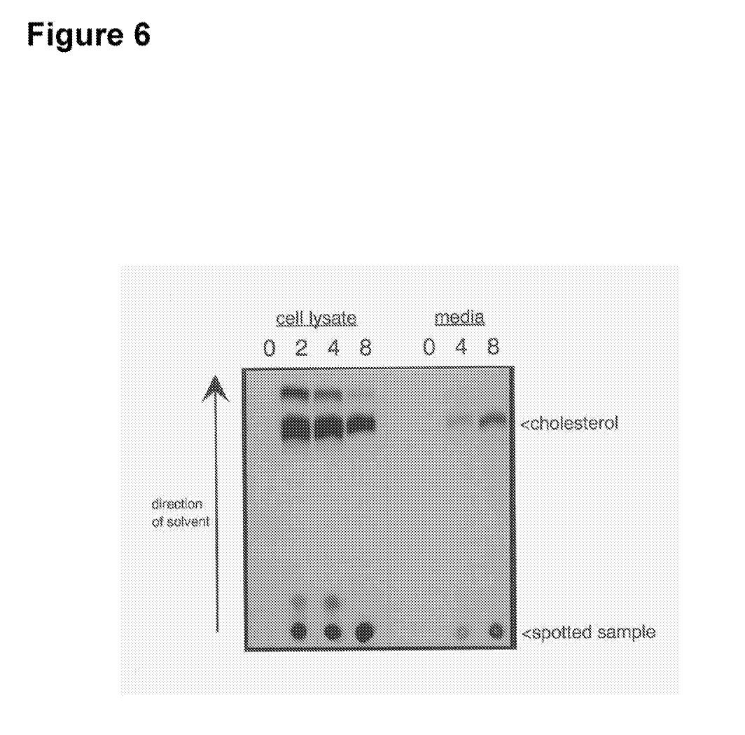

The ability of an agent to induce ER stress can also be measured indirectly by virtue of an increase in cholesterol accumulation in the cell. Cholesterol accumulation can be detected using any standard method. Increased de novo cholesterol biosynthesis can also be detected using any standard technique, e.g. by following the incorporation of ¹⁴C-acetate (New England Nuclear; NEN) into cholesterol and cholesterol derivatives. Labeled cholesterol products are then resolved by, e.g., thin layer chromatography (TLC) and quantified by scintillation counting, as shown in FIG. 6.

Virtually any agent can be tested in such an assay, including, but not limited to, natural or synthetic nucleic acids, natural or synthetic polypeptides, natural or synthetic lipids, natural or synthetic small organic molecules, and the like. In one preferred format, test agents are provided as members of a combinatorial library. In preferred embodiments, a collection of small molecules are tested for the ability to modulate the expression or activity of an ER stress related gene or gene product. A “small molecule” refers to any molecule, e.g., a carbohydrate, nucleotide, amino acid, oligonucleotide, oligopeptide, lipid, inorganic compound, etc. that can be tested in such an assay. Such molecules can modulate the expression or activity of any of the ER stress related genes or gene products by any of a number of mechanisms, e.g., by binding to a promoter and modulating the expression of the encoded protein, by binding to an mRNA and affecting its stability or translation, or by binding to a protein and competitively or non-competitively affecting its interaction with, e.g., other proteins in the cell. Further, such molecules can affect the ER stress related protein directly or indirectly, i.e., by affecting the expression or activity of a regulatory of the protein. Preferably, such “small molecule inhibitors” are smaller than about 10 kD, preferably 5, 2, or 1 kD or less.

As discussed above, test agents can be screened based on any of a number of factors, including, but not limited to, a level of a polynucleotide, e.g., mRNA, of interest, a level of a polypeptide, the degree of binding of a compound to a polynucleotide or polypeptide, the intracellular localization of a polynucleotide or polypeptide, any biochemical properties of a polypeptide, e.g., phosphorylation or glycosylation, or any functional properties of a protein, such as the ability of the protein to induce the expression of other genes or to induce cholesterol biosynthesis. Such direct and indirect measures of protein activity in vivo can readily be used to detect and screen for molecules that modulate the activity of the protein.

(a) Combinatorial Libraries

In certain embodiments, combinatorial libraries of potential modulators will be screened for an ability to bind to a polypeptide or to modulate the activity of the polypeptide. Conventionally, new chemical entities with useful properties are generated by identifying a chemical compound (called a “lead compound”) with some desirable property or activity, e.g., GRP78/BiP activating activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al (1994) J. Med. Chem. 37(9): 1233-1251).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prof. Res., 37: 487-493, Houghton et al. (1991) Nature, 354: 84-88), peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al, (1992) J. Amer. Chem. Soc. 114: 9217-9218), analogous organic syntheses of small compound libraries (Chen et al (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083), antibody libraries (see, e.g., Vaughn et al. (1996) Nature Biotechnology, 14(3): 309-314), and PCT/US96/10287), carbohydrate libraries (see, e.g., Liang et al., (1996) Science, 274: 1520-1522, and U.S. Pat. No. 5,593,853), and small organic molecule libraries (see, e.g., benzodiazepines, Baum (1993) C&EN, January 18, page 33; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

(b) High Throughput Screening

Any of the assays to identify compounds capable of modulating the expression or activity of any of the genes or gene products described herein, or of otherwise modulating ER stress, are amenable to high throughput screening.

High throughput assays for the presence, absence, quantification, or other properties of test agents on cells are well known to those of skill in the art. Similarly, binding assays and reporter gene assays are similarly well known. Thus, for example, U.S. Pat. No. 5,559,410 discloses high throughput screening methods for proteins, U.S. Pat. No. 5,585,639 discloses high throughput screening methods for nucleic acid binding (Lie., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

D. Administration of ER Stress or Stress Response-Inhibiting Compounds

In numerous embodiments of the present invention, an ER stress modulating compound, i.e. a polynucleotide, polypeptide, test agent, or any compound that increases levels of GRP78/BiP mRNA, polypeptide and/or protein activity, or that decreases the level or activity of an ER stress response protein, will be administered to a mammal. Such compounds can be administered by a variety of methods including, but not limited to, parenteral, topical, oral, or local administration, such as by aerosol or transdermally, for prophylactic and/or therapeutic treatment. The pharmaceutical compositions can be administered in a variety of unit dosage forms depending upon the method of administration. For example, unit dosage forms suitable for oral administration include, but are not limited to, powder, tablets, pills, capsules and lozenges. It is recognized that the modulators (e.g., antibodies, antisense constructs, ribozymes, small organic molecules, etc.) when administered orally, must be protected from digestion. This is typically accomplished either by complexing the molecule(s) with a composition to render it resistant to acidic and enzymatic hydrolysis, or by packaging the molecule(s) in an appropriately resistant carrier, such as a liposome. Means of protecting agents from digestion are well known in the art.

The compositions for administration will commonly comprise an ER-stress modulator dissolved in a pharmaceutically-acceptable carrier, preferably an aqueous carrier. A variety of aqueous carriers can be used, e.g. buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th d., Mack Publishing Company, Easton, Pa. (1980).

The compositions containing modulators of ER stress can be administered for therapeutic or prophylactic treatments. In therapeutic applications, compositions are administered to a patient suffering from a disease (e.g. atherosclerosis) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of an ER stress modulator that is capable of preventing or slowing the development of the disease or condition in a mammal is referred to as a “prophylactically effective dose.” The particular dose required for a prophylactic treatment will depend upon the medical condition and history of the mammal, the particular disease or condition being prevented, as well as other factors such as age, weight gender, etc. Such prophylactic treatments may be used, e.g. in a mammal who has previously had the disease or condition to prevent a recurrence of the disease or condition, or in a mammal who is suspected of having a significant likelihood of developing the disease or condition.

It will be appreciated that any of the present ER stress-inhibiting compounds can be administered alone or in combination with additional ER stress-inhibiting compounds or with any other therapeutic agent, e.g., other anti-atherosclerotic or other cholesterol-reducing agents or treatments.

IV. Diagnosing Cholesterol-Associated Diseases or Conditions

In numerous embodiments, the level of ER stress in cells of a mammal will be detected, where an elevated level of ER stress in the cells compared to a value expected of control cells, or the presence of ER stress in more cells than expected in a control sample, indicates an increased level of cholesterol in the cells. This elevated level of cholesterol is, alone or in combination with other information, used to diagnose a cholesterol-associated disease or condition, or the likelihood of the mammal to develop a cholesterol-associated disease or condition.

The presence of ER stress can be detected in any of a number of ways, using methods well known to those of skill in the art. In preferred embodiments, the presence of ER stress is detected by virtue of the presence or activity of one or more genes or gene products that are expressed or activated in response to ER stress, such as any of the ER resident chaperones described herein, a SREBP, a NFκB transcription factor, and other transcription factors (e.g. GADD153, ATF-3, ATF-6, ATF4) can be used. Such genes or gene products can be detected, in vitro or in vivo, using standard methods such as immunoassays, PCR and other amplification-based methods, Northern blots, and the like.

The expression or activity of the herein-described genes and gene products can be detected in any biological sample taken from, or present in, a mammal. Preferably, the biological sample will contain cells involved in the development of a cholesterol-associated disease, such as endothelial cells, macrophages, smooth muscle cells, or hepatic cells, but can be any sample including, but not limited to, blood, urine, saliva, buccal or other samples, including tissue biopsies. In preferred embodiments, a secreted protein that is induced, directly or indirectly, by ER stress, will be detected, thereby allowing the easy detection of the protein in any of a number of samples. The determination of optimal biological sample for analysis will depend on a variety of factors, e.g., the particular condition being investigated, and can readily be determined by one of skill in the art.

It will be appreciated that any of the cholesterol-associated diseases or conditions, or the determination of a propensity to develop of any the cholesterol-associated diseases or conditions, can be accomplished using the methods of this invention alone, in combination with other methods, or in light of other information regarding the state of health of the animal.

A. Detection of Expressed Protein or Polynucleotides

In numerous embodiments of this invention, any of a number of cholesterol-associated diseases or conditions, e.g., atherosclerosis, or a propensity for a mammal to develop a cholesterol-associated disease or condition, is detected by detecting ER stress, or an ER stress response, in cells of the mammal. Because of the herein-described causative link between ER stress, e.g., as induced by elevated levels of homocysteine, and cholesterol accumulation, the detection of ER stress can be used as an indicator of cholesterol accumulation, and hence for the presence of, or a likelihood to develop, any of a number of cholesterol-associated diseases or conditions.

1. Detecting ER Stress Induced Polypeptides

ER stress related polypeptides can be detected and quantified by any of a number of means well known to those of skill in the art. These include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In a preferred embodiment, an ER-stress related polypeptide is detected using an immunoassay such as an ELISA assay (see, e.g., Crowther, John R. ELISA Theory and Practice, Humana Press: New Jersey, 1995). As used herein, an “immunoassay” is an assay that utilizes an antibody to specifically bind to the analyte (i.e., the polypeptide). The immunoassay is thus characterized by detection of specific binding of a polypeptide to an antibody specific to the polypeptide.

In an immunoassay, a polypeptide can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition, Enzyme Immunoassay (Maggio, ed., 1980); and Harlow & Lane, supra.

Immunoassays typically rely on direct or indirect labeling methods to detect antibody-analyte binding. For example, an anti-GRP78/BiP antibody can be directly labeled, thereby allowing detection. Alternatively, the anti-GRP78/BiP antibody may itself be unlabeled, but may, in turn, be bound by a labeled third antibody specific to antibodies of the species from which the second antibody is derived. The second or third antibodies can also be modified with a detectable moiety, e.g. as biotin, to which a third labeled molecule can specifically bind, such as enzyme-labeled streptavidin. Also, other antibody-binding molecules can be used, e.g., labeled protein A or G (see, generally Kronval, et al. (1973) J. Immunol., 111: 1401-1406, and Akerstrom (1985) J. Immunol., 135: 2589-2542).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Immunoassays for detecting a polypeptide can be competitive or noncompetitive. Noncompetitive immunoassays arm assays in which the amount of captured analyte is directly measured. In a preferred embodiment, “sandwich” assays will be used, for example, wherein antibodies specific for the analyte are bound directly to a solid substrate where they are immobilized. These immobilized antibodies then capture the protein of interest present in a test sample. The protein thus immobilized is then bound by a labeling agent, such as a second specific antibody bearing a label.

In competitive assays, the amount of protein present in a sample is measured indirectly, e.g., by measuring the amount of added (exogenous) protein displaced (or competed away) from a specific antibody by protein present in a sample. For example, a known amount of labeled GRP78/BiP polypeptide is added to a sample and the sample is then contacted with an anti-GRP78/BiP antibody. The amount of labeled GRP78/BiP polypeptide bound to the antibody is inversely proportional to the concentration of GRP78/BiP polypeptide present in the sample.

Any of a number of labels can be used in any of the immunoassays of this invention, including fluorescent labels, radioisotope labels, or enzyme-based labels, wherein a detectable product of enzyme activity is detected (e.g., peroxidase, alkaline phosphatase, β-galactosidase, etc.).

One of skill in the art will appreciate that it is often desirable to minimize nonspecific binding in immunoassays. Particularly, where the assay involves an antigen or antibody immobilized on a solid substrate it is desirable to minimize the amount of nonspecific binding to the substrate. Means of reducing such nonspecific binding are well known to those of skill in the art. Typically, this technique involves coating the substrate with a proteinaceous composition. In particular, protein compositions such as bovine serum albumin (BSA), nonfat powdered milk, and gelatin are widely used.

Methods of producing polyclonal and monoclonal antibodies that react specifically with a protein are known to those of skill in the art (see, e.g., Coligan, Current Protocols in Immunology (1991); Harlow & Lane, supra, Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al, Science 246:1275-1281 (1989); Ward et al, Nature 341:544-546 (1989)).

A number of peptides or a full length protein may be used to produce antibodies specifically reactive with a protein of interest. For example, recombinant protein can be expressed in eukaryotic or prokaryotic cells and purified using standard methods. Recombinant protein is the preferred immunogen for the production of monoclonal or polygonal antibodies. Alternatively, a synthetic peptide derived from any amino acid sequence can be conjugated to a carrier protein and used as an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the protein. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10′ or greater are selected and tested for their cross reactivity against non-specific proteins or even other related proteins from other organisms, using a competitive binding immunoassay. Specific polygonal antisera and monoclonal antibodies will usually bind with a K_{d} of at least about 0.1 mM, more usually at least about 1 μM, preferably at least about 0.1 μM or better, and most preferably, 0.01 μM or better.

2. Detection of ER Stress Related Polypeptides

(a) Direct Hybridization-Based Assays

Methods of detecting and/or quantifying the level of a gene transcript using nucleic acid hybridization techniques are known to those of skill in the art (see, Sambrook et al., (1989) Molecular Cloning: A Laboratory Manual, 2d Ed., vols 1-3, Cold Spring Harbor Press, New York).

For example, one method for evaluating the presence, absence, or quantity of an ER response-associated cDNA involves a Southern Blot as described above. Briefly, the mRNA is isolated using standard methods and reverse transcribed to produce cDNA. The cDNA is then optionally digested, run on a gel, and transferred to a membrane. Hybridization is then carried out using nucleic acid probes specific for the cDNA and detected using standard techniques (see, e.g., Sambrook et al., supra).

Similarly, a Northern blot may be used to detect an mRNA directly. In brief, in a typical embodiment, mRNA is isolated from a given biological sample, electrophoresed to separate the mRNA species, and transferred from the gel to a nitrocellulose membrane. As with the Southern blots, labeled probes are then hybridized to the membrane to identify and/or quantify the mRNA.

(b) Amplification-Based Assays

In another preferred embodiment, a transcript (e.g. mRNA) is detected using amplification-based methods (e.g., RT-PCR). RT-PCR methods are well known to those of skill (see, e.g., Ausubel et al., supra). Preferably, quantitative RT-PCR is used, thereby allowing the comparison of the level of mRNA in a sample with a control sample or value.

V. Kits for Use in Diagnostic and/or Prognostic Applications.

For use in diagnostic, research, and therapeutic applications suggested above, kits are also provided by the invention. In the diagnostic and research applications such kits may include any or all of the following: assay reagents, buffers, ER stress-response associated nucleic acids or antibodies, hybridization probes and/or primers, antisense polynucleotides, ribozymes, dominant negative polypeptides or polynucleotides, small molecules inhibitors of ER stress response proteins, etc. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The following non-limiting examples are illustrative of the present invention:

### EXAMPLES

### Example 1

### A. Effect of Homocysteine on the Expression of Enzymes within the Cholesterol Biosynthetic Pathway

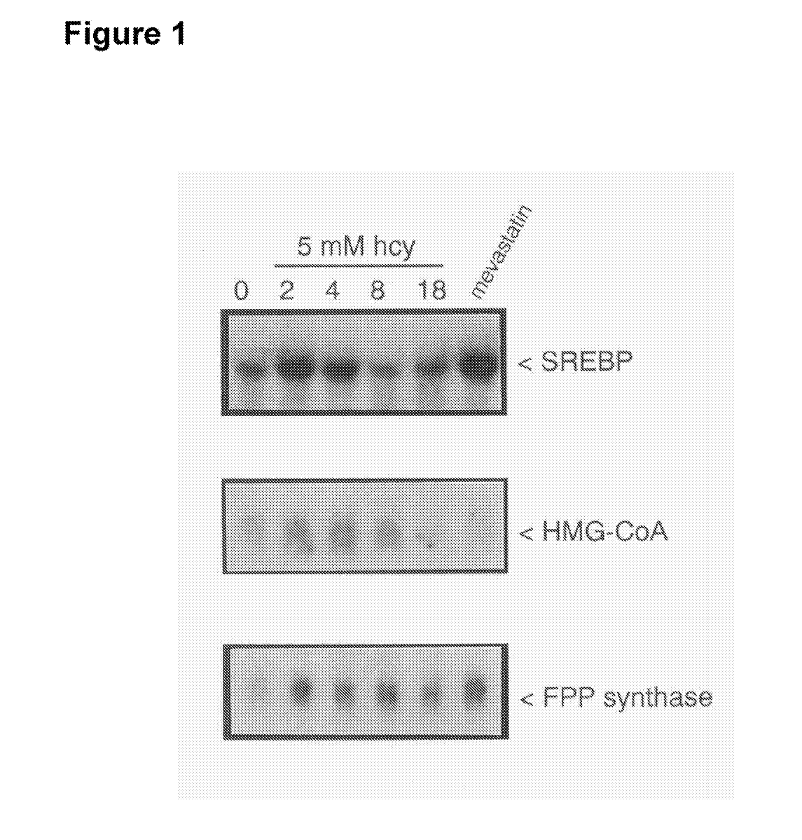

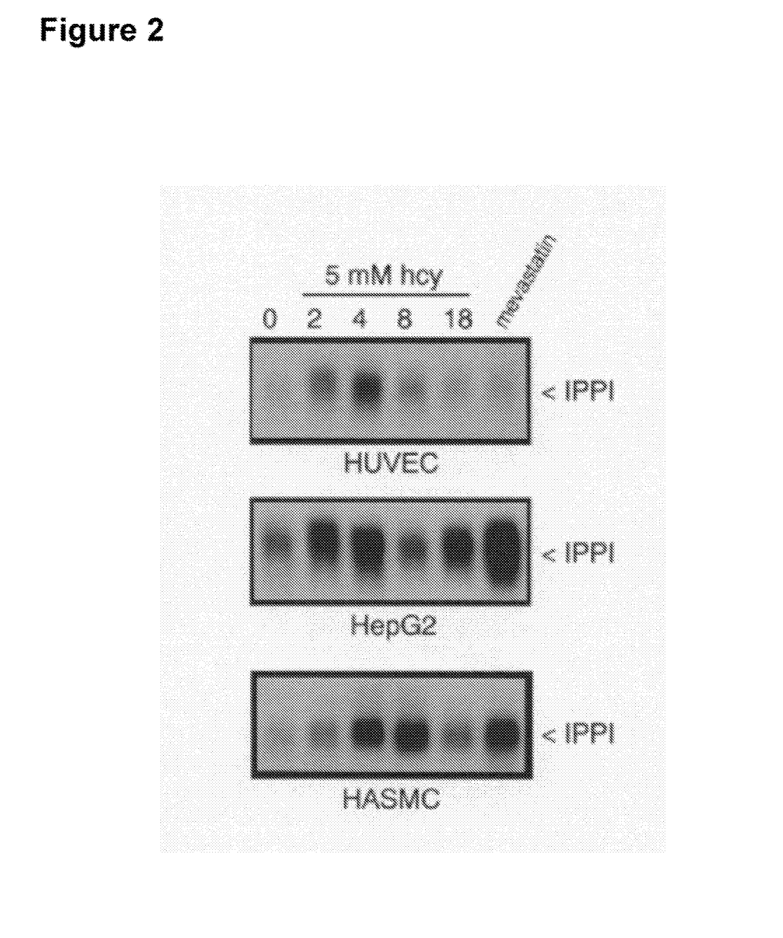

Differential display, cDNA microarrays and Northern analysis were used to investigate changes in the pattern of human umbilical vein endothelial cell (HUVEC) gene expression in the presence of elevated levels of homocysteine. Among the observed effects is an up-regulation of several genes encoding key enzymatic components of the cholesterol biosynthetic pathway, including 3-hydroxy-3-methylglutaryl coenzyme A (HMG-CoA) reductase, isopentyl diphosphate:dimethylallyl diphosphate isomerase (IPPI), and farnesyl diphosphate (FPP) synthase. The expression of clusterin (apolipoprotein J), a multifunctional protein thought to be involved in cholesterol export from foam cells and the sterol regulatory element-binding protein (SREBP), an enhancer of the cholesterol, fatty acid and triglyceride biosynthetic pathways and low-density lipoprotein (LDL) receptor gene expression, were also increased. Expression of these genes was enhanced when cells were exposed to 1-5 mM homocysteine for as little as 2 hours. It has been discovered that homocysteine induces the expression of this same set of genes in a human hepatic cell line (HepG2) and in human aortic smooth muscle cells (HASMC), although the timing, degree and endurance of the induction appears to vary with cell type (see, FIGS. 1 and 2).

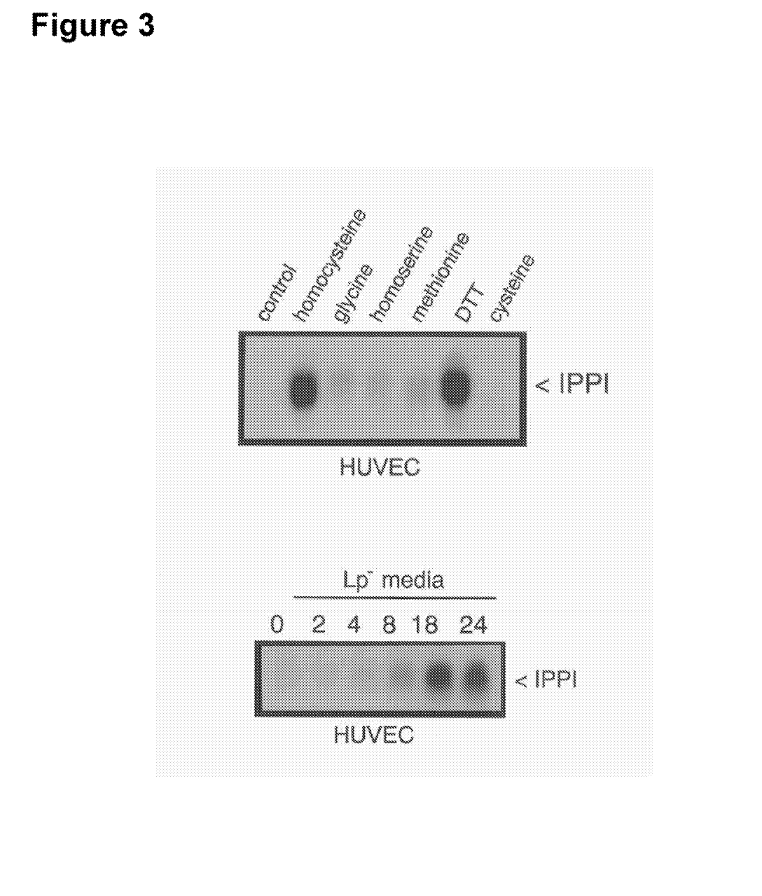

To examine the specificity of the homocysteine effect on the cholesterol biosynthetic pathway, HUVEC and HepG2 cells were treated with amino acids similar in structure to homocysteine, and the expression of cholesterol biosynthetic enzymes was monitored by Northern analysis. In contrast to homocysteine, no other amino acids, including thiol-containing methionine and cysteine, have significant effects on the expression of these genes (FIG. 3). This result suggests that the up-regulation of the cholesterol biosynthetic pathway is homocysteine-specific.

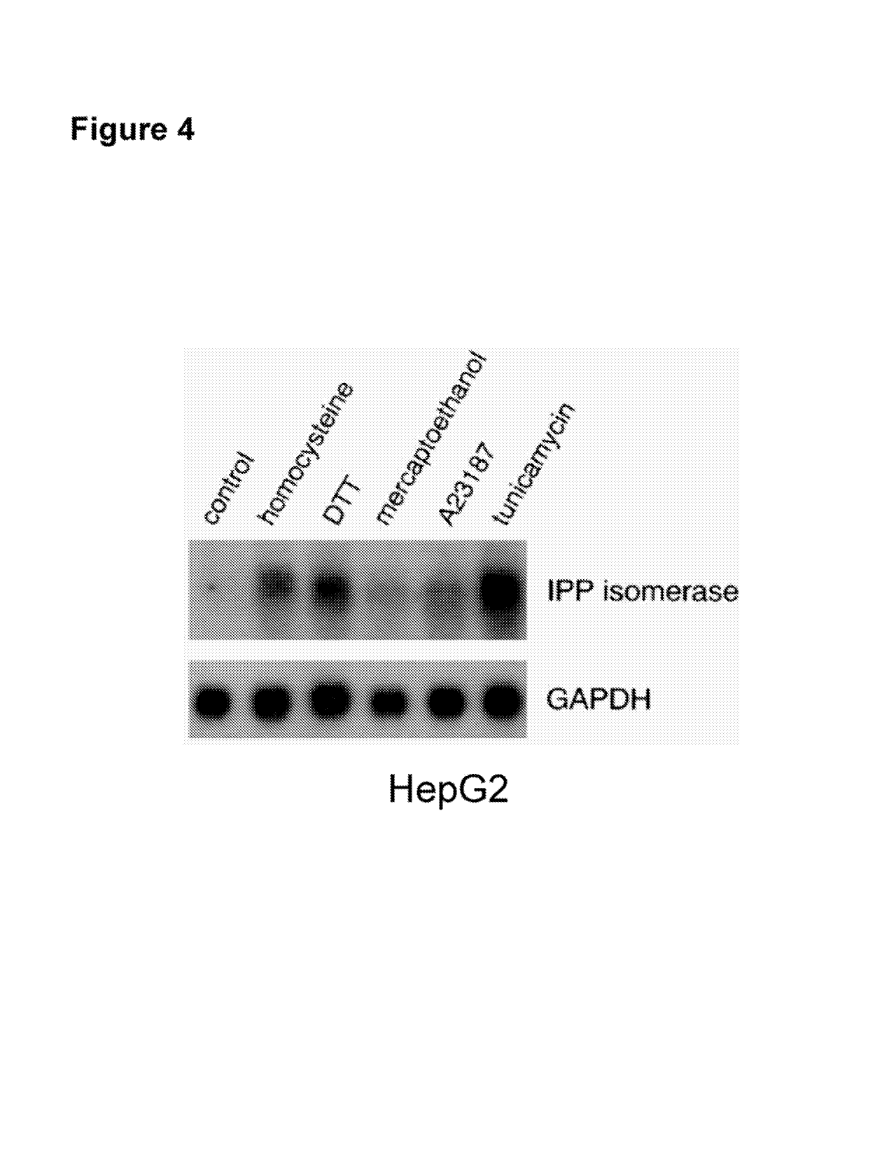

To investigate the role that ER stress plays in regulating the expression of the cholesterol biosynthetic genes, HUVEC and HepG2 cells were treated with agents known to adversely affect ER function, including tunicamycin, dithiothreitol, and the Ca²⁺ ionophore, A23187. These ER pertubants were found to induce the cholesterol biosynthetic pathway in a manner similar to that of homocysteine (FIG. 4).

B. Effect of Homocysteine on Cholesterol Biosynthesis and/or Accumulation

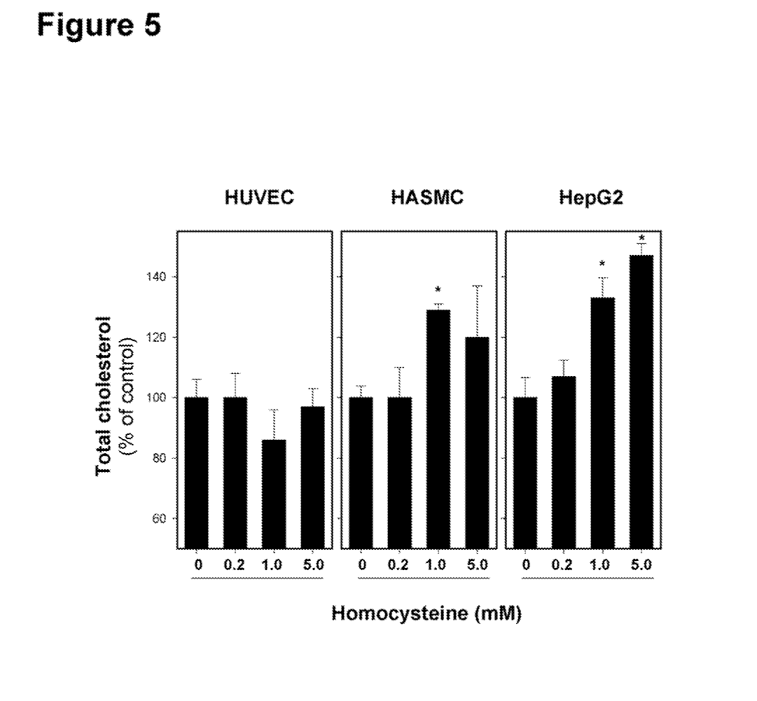

The homocysteine-dependent increase in the expression of cholesterol biosynthetic enzymes suggests that there is a corresponding induction of endogenous cholesterol production. In order to measure the effect of homocysteine on total cellular cholesterol, cells were cultured in the presence of 0-5 mM homocysteine for 24-48 h. Total cholesterol was measured and normalized to the protein content of the cells (FIG. 5). These results indicate that homocysteine promotes cholesterol accumulation in HepG2 and HASMC. There appears to be no significant change in the total cholesterol concentration of HUVEC despite the observed induction of the cholesterol biosynthetic pathway. This result suggests that HUVEC compensate for increased endogenous cholesterol accumulation by blocking cholesterol influx, and/or increasing cholesterol efflux. Homocysteine-induced cholesterol accumulation in cultured HASMC and hepatocytes may reflect HH-associated lipid accumulation in the liver and atherosclerotic lesions.

In order to measure de novo biosynthesis and the subsequent export of cholesterol from cultured cells, a sensitive cholesterol assay was used. This assay follows the incorporation of [¹⁴C]-acetate (NEN) into cholesterol and cholesterol derivatives. Labeled cholesterol products are resolved by thin layer chromatography (TLC) and quantified by scintillation counting (FIG. 6).

C. Effect of Homocysteine on LDL Binding

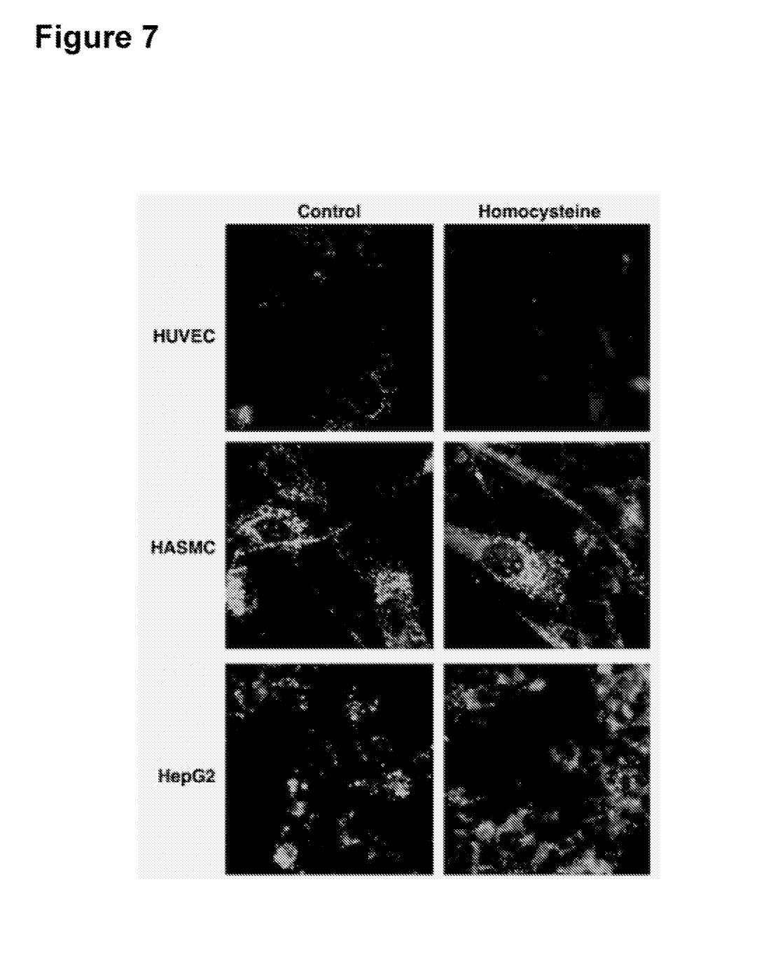

It is possible that homocysteine induces endogenous cholesterol biosynthesis in cells by blocking their ability to import cholesterol from LDL. To explore this potential mechanism, the effect of homocysteine on the ability of cells to bind fluorescently labeled LDL or acetylated (Ac) LDL (Molecular Probes Inc., Eugene, Oreg.) was examined. It was discovered that a 4 hour pre-treatment with 5 mM homocysteine has no significant effect on LDL or AcLDL binding by HUVEC (not shown). Thus, the induction of the cholesterol biosynthetic pathway, which peaks after 2-4 hours of homocysteine treatment FIG. 1-4) is not a response to cholesterol starvation. This result is consistent with the observation that endogenous cholesterol biosynthesis is not induced until cells are cholesterol starved for at least 8 h in lipoprotein-depleted media (FIG. 3). However, after 8 h incubation with 5 mM homocysteine, HUVEC exhibit a significant decrease in LDL and AcLDL binding (FIG. 7). It is hypothesized that homocysteine-induced, endogenous cholesterol production triggers the sterol-mediated feedback control mechanism in HUVEC which, in turn, inhibits further cholesterol import (i.e. LDL binding). Significantly, there is no impairment in the ability of HASMC to bind LDL even after 18 h of incubation, and our results suggest that exposure to homocysteine may further enhance LDL binding in HepG2 cells FIG. 5). These results may explain why hepatocytes and smooth muscle cells accumulate cholesterol and HUVEC do not.

D. Cholesterol Levels in CBS-Deficient Mice Having HH

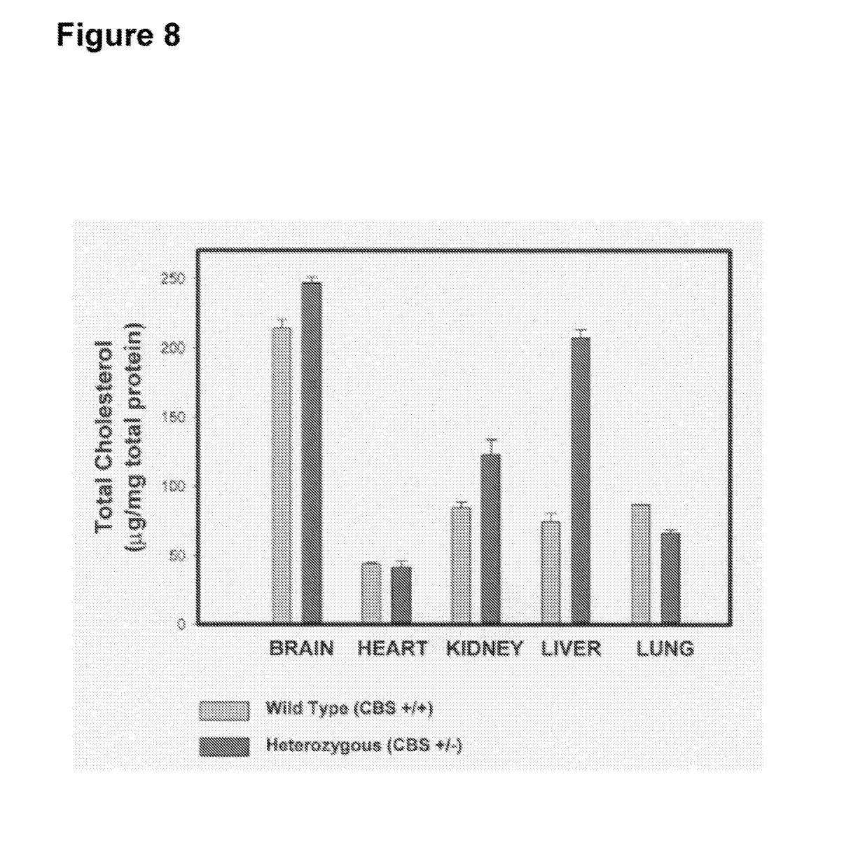

To determine the effect of elevated homocysteine levels on cholesterol biosynthesis and accumulation in vivo, experiments were performed using cystathionine synthase (CBS)-deficient mice. Tissues from heterozygous CBS-deficient and age matched control mice fed identical diets (normal mouse chow) were obtained from Dr. Nobouyo Maeda (University of North Carolina). Total cholesterol was extracted from specific tissues and determined, relative to total protein concentration FIG. 8). Our results indicate that that X specific tissues (liver, kidney, brain) of the CBS-deficient mice exhibit significant cholesterol accumulation relative to age-matched controls. Other tissues (heart and lung) showed no significant difference in cholesterol concentration. Cholesterol accumulation was most pronounced in the CBS-deficient mouse livers (2.5-fold above control). This result is consistent with the observation that these mice exhibit liver hypertrophy with hepatocytes that are enlarged, multinucleated and filled with microvesicular lipid droplets. A similar condition is found in virtually all human patients with homocystinuria.

E. Homocysteine does not Increase Cholesterol Gene Expression in Cultured Cells Resistant to ER Stress

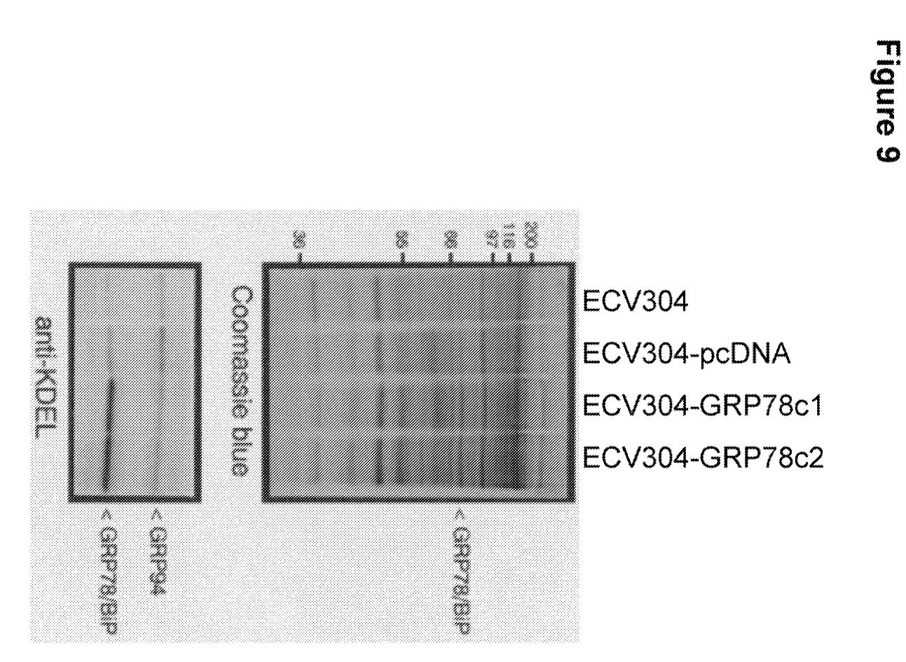

The mammalian cell expression vector, pcDNA3.1(+) containing the open reading frame of human GRP78/BiP was transfected into ECV304 cells and G418-resistant colonies were selected. These stable cell lines and their vector-transfected counterpart were maintained in ECV medium containing 800 μg/ml G418 and analyzed for GRP78/BiP expression by Western blot analysis using an anti-KDEL mAb which recognizes both GRP78/BiP and GRP94. As shown in FIG. 9, two independently isolated G418-resistant cell lines, C1 and C2 (designated ECV304-GRP78c1 and c2, respectively), had a significant increase in GRP78/BiP protein levels (approximately 4-fold), compared to either wild-type (ECV304) or vector-transfected ECV304 cells (ECV304 pcDNA). In contrast to GRP78/BiP, GRP94 protein levels were unchanged in these cell lines (FIG. 1), suggesting that alterations in GRP78/BiP protein levels do not affect endogenous GRP94 protein levels.

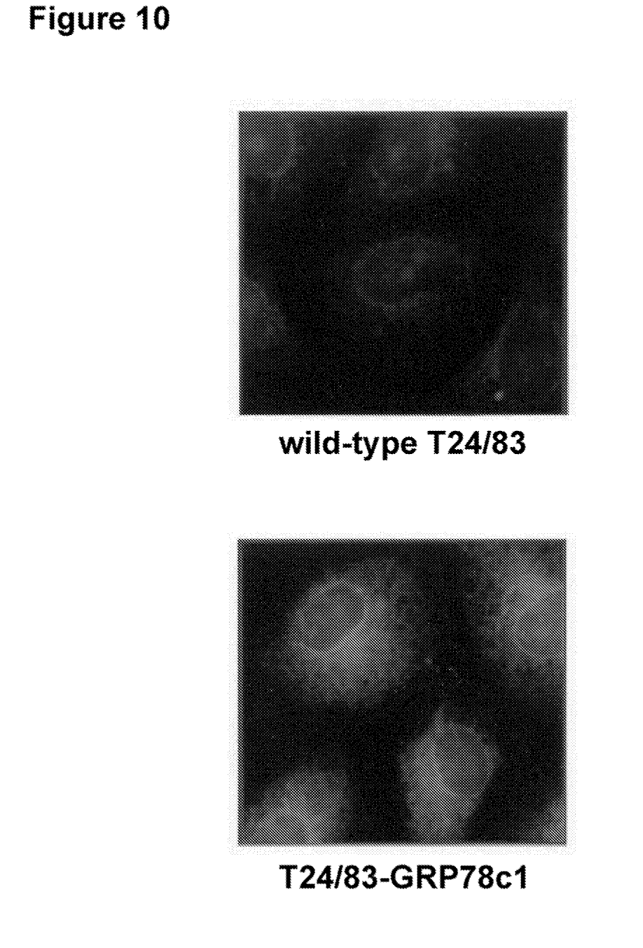

To examine the cellular localization of GRP78/BiP, ECV304 cells cultured on coverslips were examined by indirect immunofluorescence using an anti-GRP78/BiP polyclonal antibody. In wild-type cells, GRP was concentrated in the perinuclear region, consistent with its location in the endoplasmic reticulum (FIG. 10). GRP78/BiP was also localized to the ER in the ECV304-GRP78c1 cell line, but at a much greater intensity, a result consistent with the Western blot analyses. No specific staining was observed in ECV304 cells immunostained with normal mouse IgG (data not shown).

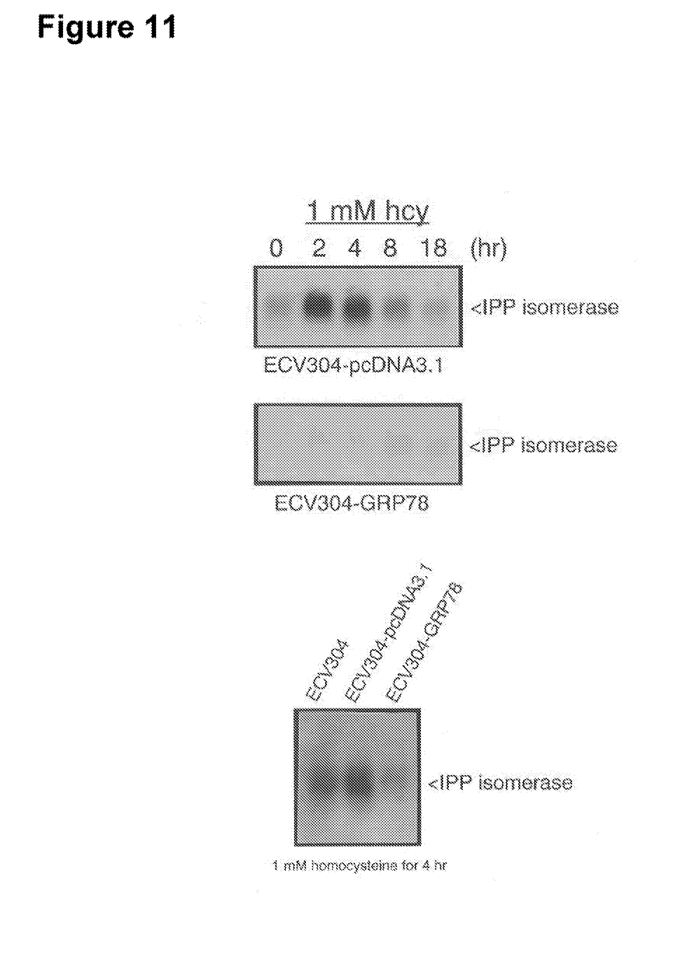

Overexpression of GRP78/BiP blocks the homocysteine-induced expression of IPPI-Vector-transfected or overexpressing GRP78/BiP ECV304 cells were treated with 5 mM homocysteine for various time periods up to 18 hr. Total RNA was isolated from these cells and Northern blot analysis was performed using a radiolabelled IPPI cDNA probe. As shown in FIG. 11, IPPI expression (a marker for the endogenous cholesterol biosynthetic pathway) was blocked in the GRP78/BiP cells, compared to the vector-transfected control cells. Given that overexpression of GRP78/BiP has been shown previously to protect cells from ER stress, these studies indicate that cellular cholesterol biosynthesis can be inhibited by alleviating ER stress.

Materials and Methods

The following materials and methods can be used for Example, as well as for any of the methods described in the present invention.

A. Cell Culture Systems

Cultured human cells relevant to the development and progression of atherosclerosis are used to investigate the mechanisms by which homocysteine enhances cholesterol biosynthesis and the role—that this process plays in the disease. The effect of elevated levels of homocysteine on the cells of the vessel wall are examined, including human umbilical vein endothelial cells (HUVEC) and human aortic smooth muscle cells (HASMC, Cascade Biologicals, Portland Oreg.). To investigate the possible role of homocysteine in the conversion of macrophages to foam cells, cholesterol biosynthesis and uptake are examined in the monoblastic cell line, U937 (American Type Culture Collection (ATCC), Manassas, Va.). These cells are utilized as monocytes and as macrophages in their differentiated form. Hepatocytes (HepG2, ATCC), the major producers of circulating cholesterol (in the form of LDL) are also studied. HUVEC, HASMC and HepG2 cells can be easily grown in the laboratory using standard methodology. Cells are grown in the presence or absence of 0 to 5 mM homocysteine for various lengths of time. As described previously, homocysteine concentrations up to 5 mM do not cause EC injury and only increase intracellular levels of homocysteine approximately 4-fold, compared to untreated cells. Controls will include cells treated with similar concentrations of cysteine, methionine and glycine.

The transformed HUVEC line, ECV304, was obtained from the American Type Culture Collection (ATCC; Rockville, Md.) and cultured in ECV medium (M199 medium containing 10% fetal bovine serum, 100 μg/ml penicillin and 100 μg/ml streptomycin) in a humidified incubator at 37° C. with 5% CO₂.

B. De Novo Cholesterol Biosynthesis

De novo cholesterol biosynthesis and export can be measured in cultured cells by monitoring the incorporation of [¹⁴C]-acetate (NEN) into [¹⁴C]-cholesterol or cholesterol derivative (Brown et al., (1978) J. Biol. Chem. 253: 1121-8; Metherall et al., (1996) J. Biol. Chem. 27: 2627-33; Rawson et al., (1998) J. Biol. Chem. 273:28261-9). Cell monolayers will be harvested in 0.2 M NaOH, and lipids extracted in hexane/isopropanol (3:2). The lipid fraction is dried in a SpeedVac Concentrator (Savant) and the sterol residue dissolved in hexane. [¹⁴C]-cholesterol and its derivatives are resolved by thin layer chromatography (TLC) on Silica Gel G plates using a petroleum ether, diethyl ether, acetic acid (60:39:1) solvent system. The dried TLC plates is exposed to Kodak X-Omat AP film for 1-3 days. Cholesterol standards/markers are visualized by staining with iodine vapour. To quantify, the regions of the TLC plate containing the signal is scraped and the silica counted in a liquid scintillation counter (Beckman LS6000LL).

1. Total Cholesterol Levels

Cultured cells or tissues are snap-frozen in liquid nitrogen and homogenized in lysis buffer containing 0.1% Triton X-100. Lipids are extracted with hexane:isopropanol (3:2), dried and resuspended in hexane. Colorimetric cholesterol assays is carried out using the Sigma Diagnostics Cholesterol Reagent (Sigma) to determine total cholesterol levels. Total plasma cholesterol are measured using the same assay but without the lipid extraction step.

C. Mouse Models of HH

Animal models of HH can be used to examine the in vivo effects of homocysteine-induced cholesterol biosynthesis and accumulation. For example, heterozygous CBS-deficient mice can be used (Watanabe et al., (1995) PNAS USA 92:1585-1589). Relative to wild-type controls, heterozygous and homozygous CBS-deficient mice typically exhibit a 2- and 50-fold increase in plasma homocysteine, respectively. Significantly, these mice suffer from fatty livers. One advantage of this system is that it better reflects the human condition of mild to moderate HH since the increase in homocysteine results from a methionine-enriched and/or vitamin-deficient diet. Another advantage is that the degree and timing of HH can be controlled though manipulations of diet and dietary supplements.

D. Statistical Analysis

Results are presented as the means ±SEM. Significance of differences between control and GRP78/BiP-overexpressing cells was determined by ANOVA. On finding significance with ANOVA, unpaired Student's t-test are performed. For all analyses, p<0.05 is considered significant

E. Generation of a Stable ECV304 Cell Lane Overexpressing GRP78/BiP

Construction of the Mammalian Expression Plasmid Encoding Human GRP78/BiP. The cDNA encoding the open-reading frame of human GRP78/BiP (approximately 1.95 kb) was amplified by reverse transcriptase-PCR using total RNA from primary HUVEC. Primers used for the reverse transcriptase-PCR procedure were synthesized at the Institute for Molecular Biology (MOBIX), McMaster University (Hamilton, ON). GRP78/BiP cDNA was generated using SuperScript RNase H-reverse transcriptase (Gibco/BRL, Burlington, ON) and a primer complimentary to a sequence in the 3′-untranslated region of the human GRP78/BiP mRNA transcript (AB10230; 5′-TAT TAC AGC ACT AGC AGA TCA GTG-3′). For PCR amplification, the forward primer AB10231 (5′-CTT AAG CTT GCC ACC ATG AAG CTC TCC CTG GTG GCC GCG-3′) contained a Kozak consensus sequence (bold) prior to the initiating ATG and a terminal HindIII restriction site (underline). The reverse primer AB10232 (5′-AGG CCT CGAG CT ACA ACT CAT CTT TTT CTG CTG T-3′) contained a terminal XhoI restriction site (underline) adjacent to the authentic termination codon of the GRP79/BiP cDNA. PCR reactions took place in a final volume of 50 PI containing 2 μl of the RT reaction, 100 ng of primers, 2.5 U Taq polymerase (Perkin-Elmer, Mississauga, ON) in a buffer consisting of 1.5 mM MgCl₂, 50 mM KCl, 10 mM Tris-HCL (pH 8.8) and 0.5 mM of each dNTP. All samples were subjected to amplification in a DNA thermal cycler 480 (Perkin-Elmer) with a step programme of 30 cycles of 94° C. for 1 min, 58° C. for 1 min, and 72° C. for 1 min. The amplified GRP78/BiP cDNA was separated on a 0.8% agarose-TBE gel containing ethidium bromide, purified from the agarose gel using the QIAEX gel extraction kit (Qiagen, Mississauga, ON) and ligated into T-ended pBluescript (KS) (Stratagene, La Jolla, Calif.). The ligation mixture was then used to transform competent DH5α cells (Gibco/BRL). Plasmids containing inserts were digested with HindIII and XhoI, and the GRP78/BiP cDNA was purified from agarose and ligated into the HindIII/XhoI site of the mammalian expression vector pcDNA3.1(+) (Invitrogen, Carlsbad, Calif.) to produce the recombinant plasmid, pcDNA3.1(+)GRP78/BiP. Authenticity of the GRP78/BiP cDNA sequence was confirmed by fluorescence-based double-stranded DNA sequencing (MOBIX). The construct was subsequently purified using QIAGEN Plasmid Midi Kits and resuspended in Tris-EDTA buffer (pH 7.4) to a concentration of 1.0 mg/ml.

Establishment of Stable ECV304 Cell Lines Overexpressing GRP78/BiP. ECV304 cells grown to 30% confluency were transfected with 5 μg of the pcDNA3.1(+)-GRP78/BiP expression plasmid using 30 PI of SuperFect Transfection reagent (Qiagen) as described by the manufacturer. As a vector control, pcDNA3.1(+) was used to transfect ECV304 under the same conditions. Stable transfectants were selected in ECV medium containing 12 mg/ml G418 (Gibco/BRL) for two weeks. G418-resistant clones were subsequently identified, isolated and cultured in ECV medium containing G418. Overexpression of GRP78/BiP was assessed using Western blotting and indirect immunofluorescence as described below.

Immunoblot Analysis. The anti-KDEL mAb (SPA-827), which recognizes both GRP78/BiP and GRP94, was purchased from StressGen Biotechnologies (Victoria, BC). Polyclonal antibodies to human GRP78/BiP were purchased from Santa Cruz Biotechnology (Santa Cruz, Calif.). Total protein lysates from ECV304 cells were solubilized in SDS-PAGE sample buffer, heated to 95° C. for 2 min, and separated on SDS-polyacrylamide gels under reducing conditions as described previously (Outinen et al., (1998), supra; Austin et al, 1995). After incubation with the appropriate primary and horseradish peroxidase (HRP)-conjugated secondary antibodies (Gibco/BRL), the membranes were developed using the Renaissance chemiluminescence reagent kit (Dupont/NEN, Mississauga, ON).

Immunohistochemistry and Image Analysis. Immunohistochemistry and image analysis for GRP78/BiP was performed as described previously (Outinen et al., 1998, supra). Images were subsequently captured and analyzed using Northern Exposure image analysis/archival software (Mississauga, ON).

Preparation of Total RNA. Total RNA was Isolated from Cells Using the Rneasy Total RNA Kit (Qiagen) and resuspended in diethyl pyrocarbonate-treated water. Quantification and purity of the RNA was assessed by A260/A280 absorption, and RNA samples with ratios above 1.6 were stored at −70° C. for further analysis.

### Example 2

Methods

Cell culture and treatment conditions. Primary human umbilical vein endothelial cells (HUVEC) were isolated by collagenase treatment of human umbilical veins (Jaffe, E. A, 1973) and cultured in EC medium (M199 medium, 20 μg/ml endothelial cell growth factor, 90 μg/ml porcine intestinal heparin, 100 μg/ml penicillin and 100 μg/ml streptomycin) containing 20% fetal bovine serum (Hyclone; Logan, Utah) in a humidified incubator at 37° C. with 5% CO₂. Cells from passages 2-4 were used in these studies. Human aortic smooth muscle cells (HASMC) were purchased from Cascade Biologicals (Portland, Oreg.) and cultured in M231 media (Cascade Biologicals) containing smooth muscle cell growth supplement (Cascade Biologicals). The human hepatocarcinoma cell line, HepG2, was obtained from the American Type Culture Collection (ATCC; Rockville, Md.) and cultured in A-DMEM containing 10% fetal bovine serum. DL-homocysteine, L-methionine, DL-cysteine, glycine, DL-dithiothreitol (DMF), tunicamycin, A23187 and β-mercaptoethanol were purchased from Sigma (St. Louis, Mo.). These compounds were prepared fresh in culture medium, sterilized by filtration and added to the cell cultures.

Determination of intracellular levels of homocysteine. HepG2 cells exposed to 1 or 5 mM homocysteine for 0 to 24 h were washed three times in DMEM media containing 10% serum and three times in 1×PBS. Cells were lysed in H₂O by three freeze/haw cycles and cellular debris removed by centrifugation. Total homocysteine (tHcy), defined as the total concentration of homocysteine after quantitative reductive cleavage of all disulfide bonds (Mudd, S. H, et al 2000), in cellular lysates was determined using the IMx System. (Abbott Laboratories, Mississauga, ON) and normalized to total protein concentration.

Hyperhomocysteinemia in mice. Heterozygous CBS-deficient mice (CBS+/−) (12) were crossbred to wild-type C57BL6J mice (CBS+/+). (The Jackson Laboratory). Genotyping for the targeted allele was performed by polymerase chain reaction (Watanabe, M., 1995). At the time of weaning, offspring were fed one of three diets: 1) a control diet that contained 7.5 mg folic acid/Kg (LM-485, Harlan Teklad); 2) a high methionine diet that was identical to the control diet except that the drinking water was supplemented with 0.5% L-methionine, or 3) a high methionine/low folate diet that contained 1.5 mg folic acid/Kg and succinylsulfathiazole (1.0 mg/Kg) and drinking water that was supplemented with 0.5% L-methionine (Lentz, S. R., 2000). After 2 to 16 weeks on experimental diet, mice were euthanized with sodium pentobarbital (75 mg ip), plasma was collected in EDTA (final concentration 5-10 mM) for measurement of tHcy, and their tissues removed and snap frozen in liquid N₂ before storage at −70° C. Plasma tHcy was measured by high performance liquid chromatography and electrochemical detection as described previously (Malinow, M. R. et al, 1990). The experimental protocol was approved by the University of Iowa and Veterans Affairs Animal Care and Use Committees.

Histological Analysis. Liver tissue was fixed in formalin, and eight μm tissue sections were stained with hematoxylin and eosin as described previously (Lentz, S. R, 1997).

Preparation of Total RNA. Total RNA was Isolated from Cells or Tissues Using the Rneasy Total RNA Kit (Qiagen, Santa Clarita, Calif.) and resuspended in diethyl pyrocarbonate (DEPC)-treated water. Quantification and purity of the RNA was assessed by A₂₆₀/A₂₈₀ absorption, and RNA samples with ratios above 1.6 were stored at −80° C. for further analysis.

Northern blot analysis. Total RNA (10 μg/lane) was resolved on 22 M formaldehyde/1.2% agarose gels and transferred overnight onto Zeta-Probe GT nylon membranes (Bio-Rad, Toronto, ON) in 10×SSC. The RNA was cross-linked to the membrane using a UV crosslinker (PDI Bioscience, Toronto, ON) prior to hybridization. Specific probes were generated by labelling the cDNA fragments with [α-³²]dCTP (NEN) using a random primed DNA labelling kit (Boehringer Mannheim, Laval, QC). After overnight hybridization at 43° C., the membranes were washed as described by the manufacturer, exposed to X-ray film and subjected to autoradiography. Changes in steady-state mRNA levels were quantified by densitometric scanning of the membranes using the ImageMaster VDS and Analysis Software (Amersham Pharmacia Biotech). To correct for differences in gel loading, integrated optical densities were normalized to human glyceraldehyde 3-phosphate dehydrogenase (GAPDH). The human IPP isomerase cDNA encodes an 837 bp DNA fragment from the 3′ untranslated region of the IPP isomerase gene. cDNA probes encoding human HMG CoA reductase and FPP synthase were kindly provided by Dr. Skaidrite Krisans (San Diego State University, San Diego, Calif.), human SREBP-1 cDNA (#AA568572) was purchased from Genome Systems (St Louis, Mo.) and LDL receptor cDNA was purchased from ATCC. The cDNA probes encoding GRP78 or GADD153 have been described previously (Outinen, P. A., et al 1998, 1999).

Immunoblot analysis. The anti-KDEL mAb (SPA-827), which recognizes both GRP78/BiP and GRP94, was purchased from StressGen Biotechnologies (Victoria, BC). The anti-SREBP-1 and -2 mAbs (clones IgG-2A4 and IgG-1C6, respectively) were purchased from PharminGen (Mississauga, ON). Total protein lysates from mouse tissues or cultured cells were solubilized in SDS-PAGE sample buffer, heated to 95° C. for 2 min, and separated on SDS-polyacrylamide gels under reducing conditions, as described previously (Outinen, P. A., et al 1998, 1999). After incubation with the appropriate primary and horseradish peroxidase (HRP)-conjugated secondary antibodies (Gibco/BRL), the membranes were developed using the SuperSignal chemiluminescent substrate (Pierce; Rockford, Ill.).

Uptake of BODIPY FL LDL and image analysis. Cells treated in the absence or presence of homocysteine were washed with PBS and incubated in media containing 10 μg/ml BODIPY FL LDL (Molecular Probes, Eugene, Oreg.). After incubation at 37° C. for 2 h, cells were washed with PBS, fixed in 3% formaldehyde in PBS, and the uptake of LDL was detected by fluorescence microscopy as described previously (Outinen, P. A., et al 1998, 1999). Images were subsequently captured and analyzed using Northern Exposure image analysis/archival software (Mississauga, ON).

Total cholesterol and triglyceride levels. Cultured cells or tissues were homogenized in lysis buffer containing 0.1% Triton X-100. Cell lysates were saponified and lipids were extracted with hexane/isopropanol (3:2) (Brown, M. S., 1978). Colorimetric cholesterol and triglyceride assays were carried out using the Sigma Diagnostics Cholesterol and Triglyceride Reagents (Sigma). Total plasma cholesterol and triglycerides were measured using the same assays but without the lipid extraction step.

Statistical analysis. Results are presented as the means ±SD. Differences in total cholesterol, triglycerides and homocysteine between wild-type mice and mice with diet-induced hyperhomocysteinemia were determined by two-way analysis of variance (ANOVA). On finding significance with ANOVA, unpaired Student's t-test were performed. For all analyses, P<0.05 was considered significant.

Results

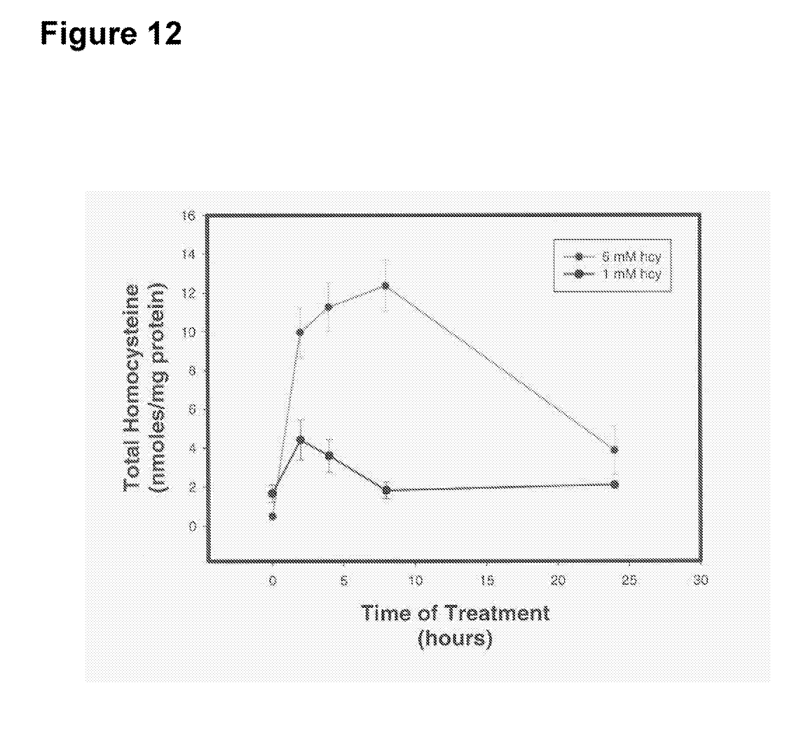

Intracellular levels of homocysteine. Previous studies have suggested that elevated intracellular levels of homocysteine cause ER stress and alter gene expression in HUVEC (Outinen, P A et al, 1998). In order to increase intracellular homocysteine levels in HepG2 cells, cells were treated with varying concentrations of DL-homocysteine up to 5 mM. FIG. 12 shows that to attain a 2 to 6 fold transient increase in intracellular homocysteine in HepG2 cells requires an extracellular homocysteine concentration of 1 to 5 mM. Extracellular homocysteine concentrations of up to 5 mM have no effect on overall cell number or viability as determined by Trypan blue and ⁵¹Cr release assays (Outinen, P A et al. 1998, 1999).

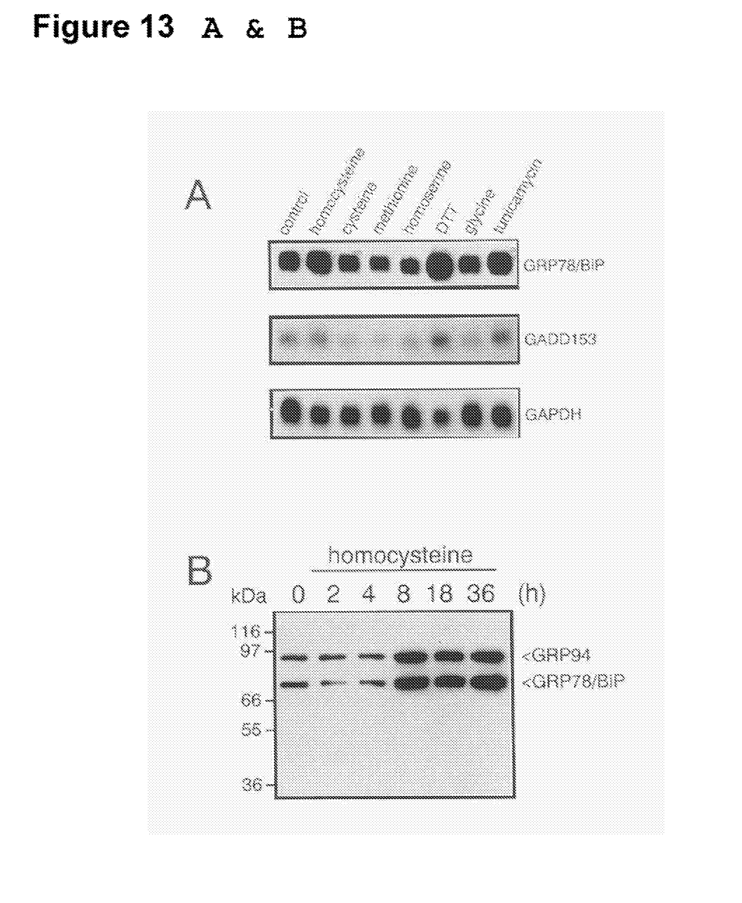

Homocysteine activates the unfolded protein response (UPR) in HepG2 cells. It has been demonstrated previously, in HUVEC, that homocysteine activates the UPR, leading to increased expression of the ER stress response genes GRP78/BiP and GADD153 (Outinen, P A et al, 1998, 1999). As shown in FIG. 13A, 5 mM homocysteine also increased steady-state mRNA levels of GRP78/BiP and GADD153 in HepG2 cells. This effect was selective for homocysteine because other structurally related amino acids such as methionine, cysteine, homoserine and glycine failed to induce the expression of these ER stress response genes. In addition to homocysteine, other agents known to activate the ER UPR, including dithiothreitol (DTT) and tunicamycin, also induced the steady-state mRNA levels of GRP78/BiP and GADD153 in HepG2 cells. Consistent with induction of the steady-state mRNA levels of GRP78/BiP by homocysteine, GRP78/BiP and GRP94 protein levels were elevated in HepG2 levels following 8, 18 and 36 h treatment with homocysteine (FIG. 13B).

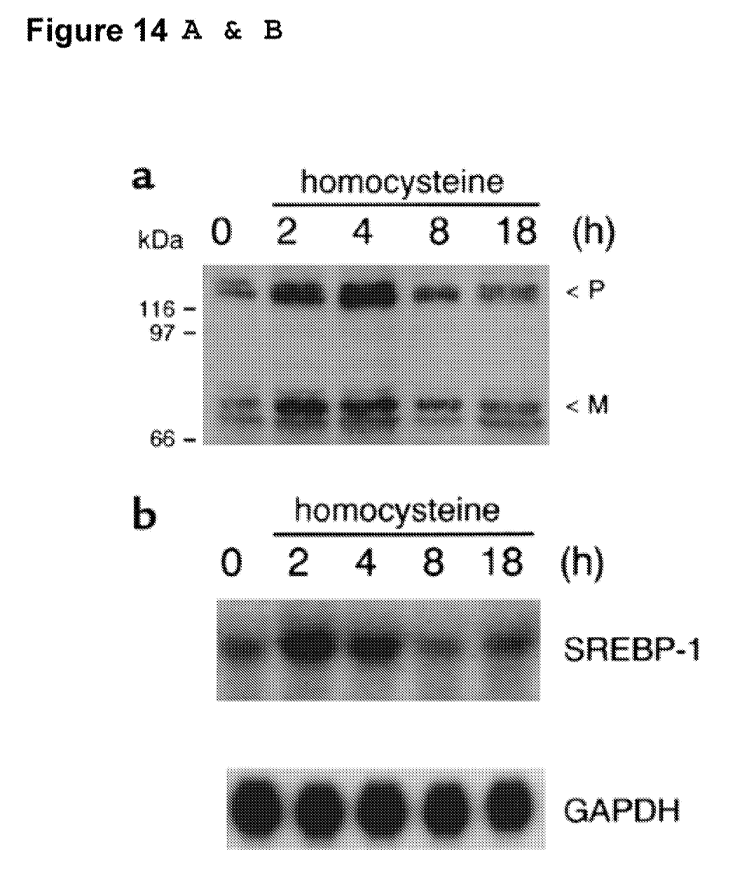

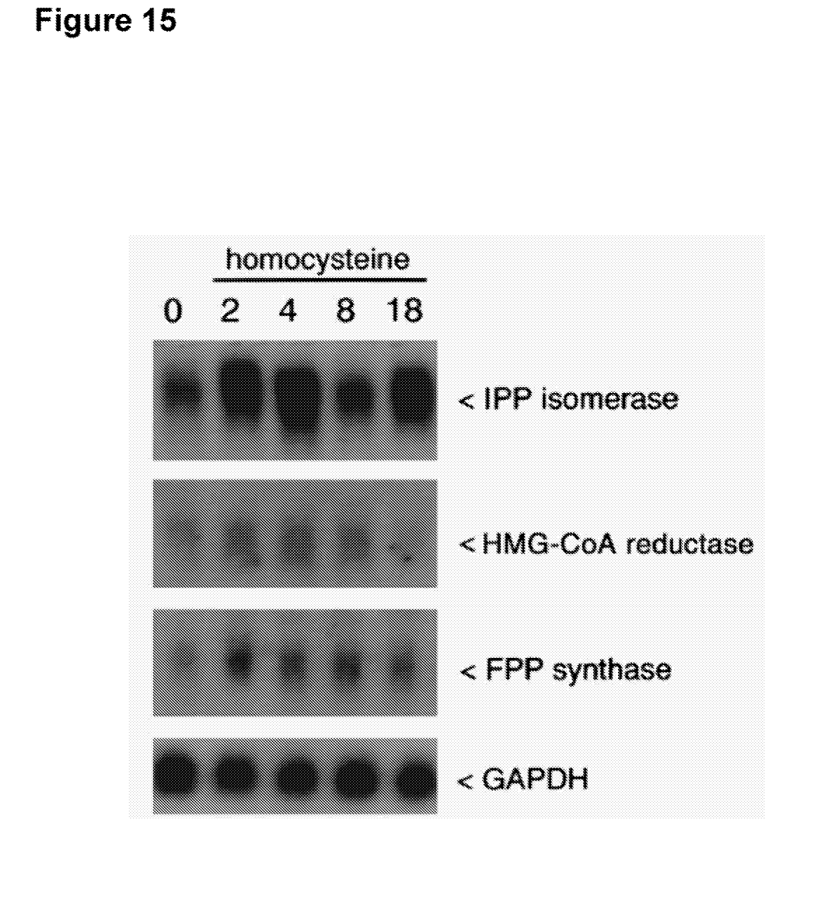

Effect of homocysteine on SREBP activation and expression of enzymes within the cholesterol biosynthesis pathway. Immunoblot analysis showed that HepG2 cells had increased levels of both active (68 kDa) and precursor (125 kDa) forms of SREBP-1 following treatment with homocysteine for 24 hours (FIG. 14A). Active and precursor forms of SREBP-2 were also increased in HepG2 cells by homocysteine (data not shown). Because activated SREBPs autoregulate their own synthesis in addition to regulating genes involved in cholesterol/triglyceride biosynthesis and uptake (Brown, M. S., and Goldstein, I. L. 1999, Horton, J. D. and Shimomura, I. 1999, Amemiya-Kudo, M., 2000), Northern blots were used to examine the effect of homocysteine on the steady-state mRNA levels of SREBP-1 and several genes encoding key enzymatic components of the cholesterol/triglyceride biosynthesis pathway. Steady-state mRNA levels of SREBP-1 were increased and peaked between 2 and 4 h following treatment with homocysteine (FIG. 14B). Furthermore, steady-state mRNA levels of genes encoding enzymes of the cholesterol biosynthetic pathway, including 3-hydroxy-3-methylglutaryl coenzyme A (HMG-CoA) reductase, isopentyl diphosphate:dimethylallyl diphosphate (IPP) isomerase, and farnesyl diphosphate (FPP) synthase, were increased and peaked between 2 and 4 hr in HepG2 cells following treatment with homocysteine (FIG. 15). The mRNA levels of genes encoding enzymes involved in fatty acid synthesis including acetyl CoA carboxylase and fatty acid synthase as well as the LDL receptor were also increased in homocysteine treated HepG2 cells (data not shown). Similar patterns of gene induction were observed in HASMC and HUVEC exposed to homocysteine (data not shown). The observation that cycloheximide does not block the induction of these genes by homocysteine (data not shown) is consistent with a mechanism involving the activation of existing precursor SREBPs (Brown, M. S. and Goldstein, J. L. 1999, Horton, J. D. and Shimomura, I. 1999).

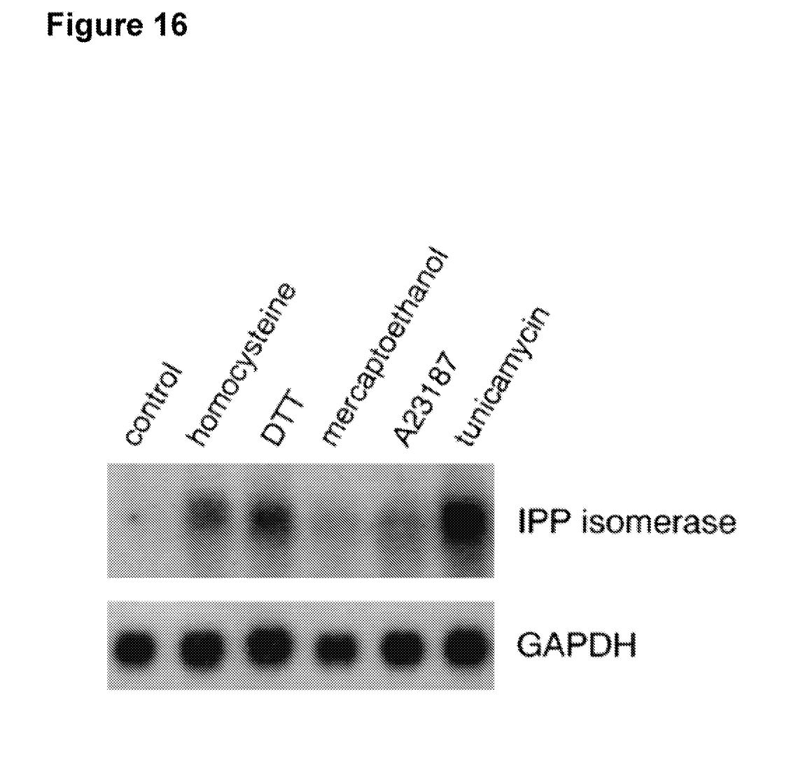

Induction of the cholesterol biosynthetic pathway involves activation of the UPR. HepG2 cells were treated with agents known activate the UPR, including tunicamycin, DTT, β-mercaptoethanol and the calcium ionophore, A23187, and Northern blot analysis was used to examine changes in IPP isomerase gene expression. To varying degrees, all of these agents, like homocysteine, induced the expression of IPP isomerase, compared with untreated cells (FIG. 16). Similar results were also observed for HASMC and HUVEC treated with homocysteine (data not shown).

Effect of homocysteine on the cellular levels of cholesterol. To determine whether the homocysteine-mediated induction of genes encoding cholesterol biosynthetic enzymes is associated with a corresponding increase in intracellular cholesterol, HepG2, HASMC and HUVEC were cultured in the absence or presence of either homocysteine or cysteine for 24-48 h, and total cholesterol and triglycerides were determined. Homocysteine, but not cysteine, increased cellular cholesterol in HepG2 and HASMC (FIG. 5). In contrast, cholesterol levels were unchanged in HUVEC, despite the increased expression of SREBP-1 and genes encoding enzymes in the cholesterol biosynthetic pathway.

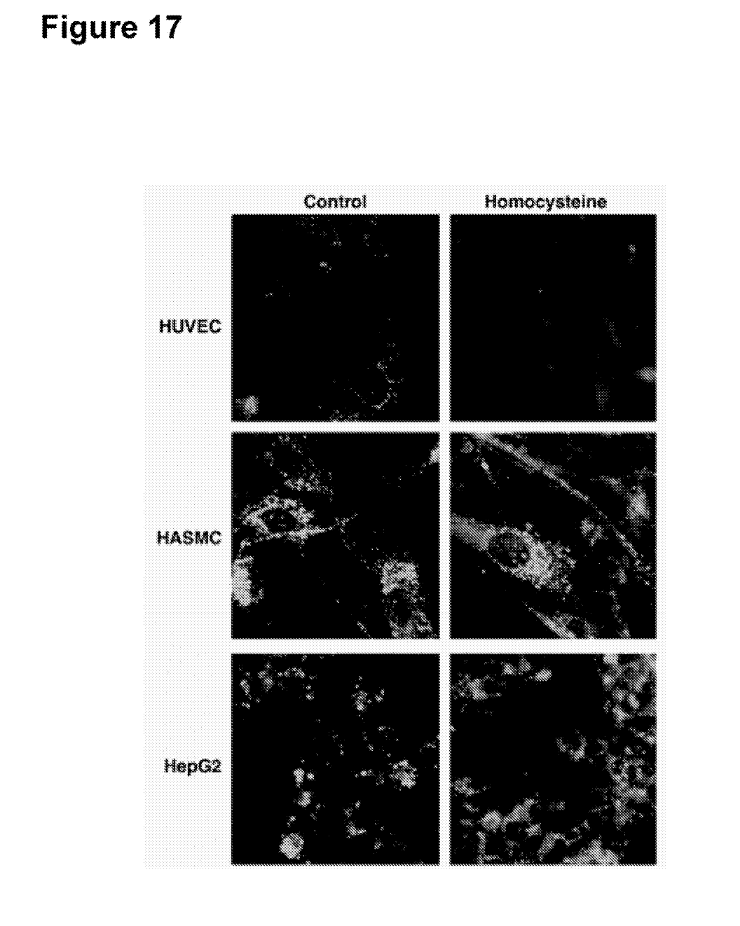

Effect of homocysteine on LDL uptake. The SREBPs are known to regulate LDL receptor expression and activity in addition to their effects on cholesterol and fatty acid biosynthesis (Brown, M. S., and Goldstein, J. L. 1999, Horton, J. D. and Shimomura, I. 1999). To explore the effect of homocysteine on cholesterol uptake via the LDL receptor, the ability of cultured cells treated with homocysteine to bind and internalize fluorescently-labelled LDL was measured (FIG. 17). The results indicate that after incubation with homocysteine, HASMC maintained their ability to endocytose LDL while HepG2 cells showed enhanced LDL uptake. In contrast, HUVEC treated with homocysteine showed a significant decrease in LDL uptake. These results indicate that the activation of the cholesterol biosynthesis pathway does not result from impaired LDL uptake in HepG2 and HASMC and may explain why these cells accumulate cholesterol and triglycerides, but HUVEC do not. Furthermore, they suggest that homocysteine modulates cholesterol uptake and accumulation in a cell specific manner.

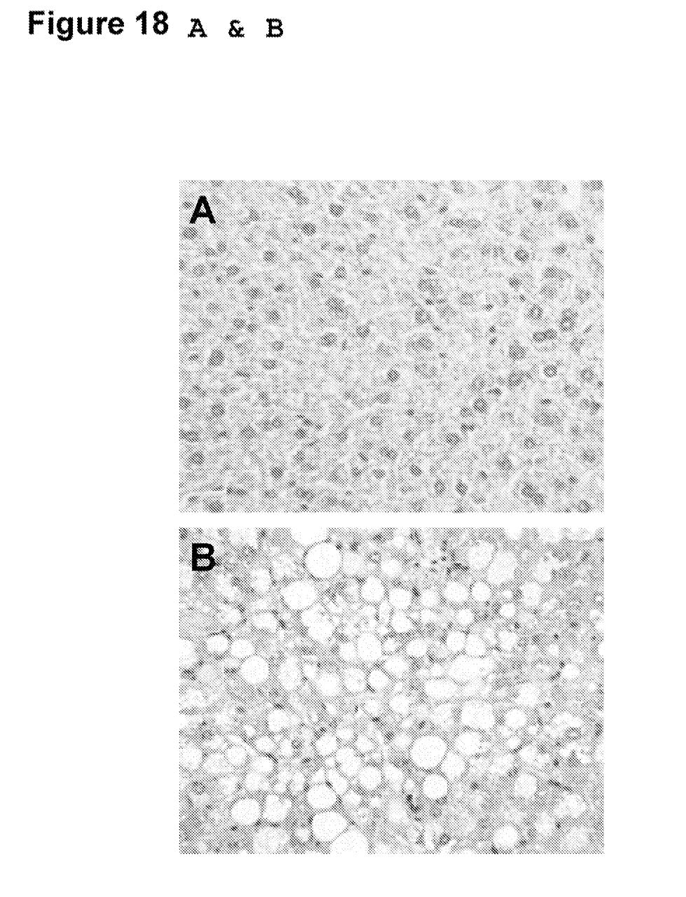

Cholesterol levels in mice with hyperhomocysteinemia. To determine the in vivo effect of hyperhomocysteinemia on lipid metabolism, cholesterol and triglyceride levels were measured in the livers and plasmas of CBS+/+ and CBS+/− mice fed control or modified (high methionine or high methionine/low folate) diets for 10-16 weeks. Compared with age-matched mice fed control diet, CBS+/+ or CBS+/− mice fed high methionine/low folate diet had markedly elevated levels of hepatic cholesterol and triglycerides (Table 1). Liver cholesterol also was elevated modestly in CBS+/+ mice fed high methionine diet Plasma cholesterol tended to be elevated in mice fed high methionine/low folate diet compared with mice fed control diet, but these differences did not reach statistical significance. No differences in plasma triglycerides were detected between groups. Compared with wild type mice fed the same diet, CBS+/− mice exhibited similar hepatic triglyceride accumulation and slightly increased cholesterol accumulation. Histological analysis of liver sections from wild type and CBS+/− mice fed high methionine/low folate diet revealed that the hepatocytes were engorged with lipid vesicles (FIG. 18). Aside from their increased levels of plasma tHcy and increased hepatic levels of cholesterol and triglycerides, all mice with diet-induced hyperhomocysteinemia appeared normal and their body weights were similar to those of mice fed control diets.

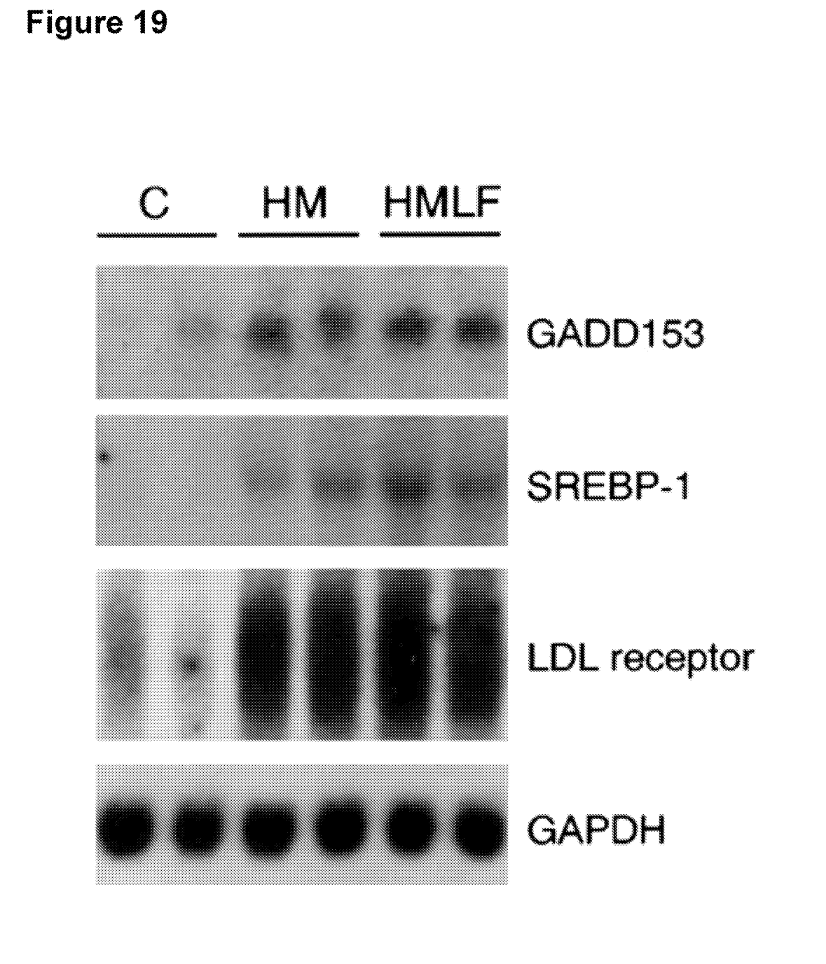

Hyperhomocysteinemic mouse liver contains increased step state levels of GADD153 and LDL receptor mRNA. To determine if hepatic cholesterol accumulation in hyperhomocysteinemic mice is associated with activation of the UPR in vivo, total RNA isolated from livers of hyperhomocysteinemic and control mice were probed for GADD153 expression (FIG. 19), an indicator of ER stress (32). Northern blot analysis demonstrated that steady state GADD153 mRNA levels were significantly higher in mice fed high methionine/low folate diets for two weeks than in control mice. This result indicates that hyperhomocysteinemia causes ER stress and UPR activation in vivo.

In addition to lipid biosynthesis, SREBPs have been shown to activate LDL receptor expression in vitro and In vivo (Brown, M. S., and Goldstein, J. L. 1999, Horton, J. D. and Shimomura, I. 1999, Horton, J. D., 1999). Northern blot analysis indicated that steady state LDL receptor mRNA levels in liver are increased in mice with diet-induced hyperhomocysteinemia compared with control mice (FIG. 19). This result is consistent with in vitro findings and suggests that a combination of increased endogenous cholesterol production along with increased LDL uptake lead to hepatic lipid accumulation in mice having diet-induced hyperhomocysteinemia

Discussion

It was previously demonstrated that elevated levels of homocysteine cause ER stress leading to activation of the UPR, in cultured human vascular endothelial cells (Outinen, P. A., et al 1998, 1999), and in the livers of homozygous CBS-deficient mice with hyperhomocysteinemia (Outinen, P. A., et al 1998). In this study, evidence is provided that the ER stress-inducing effects of homocysteine can result in dysregulated lipid biosynthesis and uptake giving rise to tissue specific cholesterol/triglyceride accumulation. Specifically, homocysteine-induced ER stress (i) activates SREBP-1 and -2, (ii) enhances expression of genes encoding enzymes within the cholesterol biosynthetic pathway and (iii) increases total cholesterol and triglyceride levels without decreasing LDL uptake in cultured HepG2 and HASMC. Consistent with the in vitro findings, livers from mice with diet-induced hyperhomocysteinemia exhibited increased levels of GADD153 mRNA and contain elevated levels of cholesterol and triglycerides.

Increased dietary methionine or deficiencies of folic acid, vitamin B6 and/or vitamin B12, which are essential cofactors involved in homocysteine metabolism, can lead to moderate hyperhomocysteinemia in humans (Selhub, J, 1993; Robinson, K et al, 1995, and Ubbink, J. B. et al, 1996) and animals (Lentz, S. R., et al, 2000; Rolland, P. H., 1995; Lentz, S. R. et al, 1996, 1997). Conditions of mild to severe hyperhomocysteinemia can be produced in wild-type or CBS-deficient mice by diets that are enriched in methionine and/or deficient in folate (Lentz, S. R., et al, 2000) (Table 1). It has been suggested that elevated plasma homocysteine promotes oxidative cytotoxic damage by increasing the production of reactive oxygen species (Wall, R. T., et al, 1980; DeGroot, P. G., 1983; Starkebaum, G. and Harlan, J. M. 1986; and Loscalzo, J. 1996). However, the oxidative stress hypothesis fails to explain why cysteine, present in plasma in concentrations 25 to 30 fold greater than homocysteine, does not also cause oxidative damage (see Jabobsen, D. W. 2000). In fact, markers of oxidative stress are not observed in cultured cells exposed to homocysteine (Outinen, P. A., et al, 1999) or in the livers of hyperhomocysteinemic mice (Eberhardt, R. T., et al. 2000). An alternative hypothesis is that cellular dysfunction is caused by elevation of intracellular concentrations of homocysteine, and that elevated plasma tHcy is a marker of increased intracellular homocysteine. To significantly increase intracellular homocysteine levels in cultured cells requires exposure to extracellular concentrations up to 5 mM or the addition of inhibitors of folate metabolism such as aminopterin (Fiskerstrand, T., Ueland, P. M. and Refsum, H. 1997). Though significantly above physiological range, 5 mM homocysteine (or 5 mM cysteine) in culture medium does not effect cell viability (Outinen, P. A., et al, 1998, 1999). However, homocysteine, but not cysteine, does cause specific intracellular effects including; inducing ER stress, activating the UPR and altering the expression of specific genes (Outinen, P. A., et al, 1998, 1999, Kokame, K., Kato, H. and Miyata, T. 1996; and, Miyata, T., Kokame, K., Agarwala, K. L. and Kato, H. 1998).

In this study, hepatic ER stress and UPR activation (demonstrated by increased steady-state levels of GADD153 mRNA) were found to be evident after two weeks in mice fed hyperhomocysteinemic diets. Significantly elevated levels of hepatic cholesterol and triglycerides were evident by 10 weeks. Plasma lipid levels, however, were relatively normal in mice with diet-induced hyperhomocysteinemia, presumably due to maintained or enhanced LDL receptor expression in liver (FIG. 19) and perhaps other tissues. These findings are consistent with previous studies demonstrating that overexpression of fully active nuclear SREBP-1a in transgenic mice leads to massive accumulation of lipids in the liver but not plasma (Horton, J. D. and Shimomura, I. 1999; and Shimano, H., et al. 1996) and perhaps explain why, with few exceptions (Li, L. J. et al, J. Cell. Physiol. 153, 575-582, 1992), epidemiological studies have not shown a correlation between elevated plasma levels of tHcy and increased plasma levels of cholesterol. The localized accumulation of lipid in tissues, such as liver, that are sensitive to ER stress may explain the prevalence of fatty liver in patients with hyperhomocysteinemia even though they have normal serum lipid profiles. These findings further highlight the importance of diet as a major contributor to the pathophysiological outcome of hyperhomocysteinemia.

Agents and/or conditions which adversely affect ER function activate the UPR, resulting in increased expression of ER chaperones such as GRP78 and 94 (Li, L J et al. 1992; and Chapman, R, et al. 1998) and transcription factors including, GADD153 and ATF6 (Wang, X. Z. et al 1998; Pahl, H. L. 1999; and Haze, K., et al. 1999). Furthermore, overexpression or misfolding of secretory proteins in mammalian cells results in a dramatic dilation of the ER. Recent studies have indicated that the UPR regulates lipid biosynthesis in yeast (Cox, J. S., et al. 1997) and dolichol biosynthesis, which is part of the cholesterol biosynthesis pathway, in human fibroblasts (Doerrler, W. T. and Lehrman, M. A. 1999). Thus, it is likely that the UPR coordinates the synthesis of ER chaperones as well as ER membrane components to increase the folding capacity and the space required to accommodate accumulation of unfolded proteins. These studies indicate that the UPR is an important cellular stress response and plays a critical role in ER biogenesis. The findings further suggest that activation of the UPR by homocysteine may allow for the overproduction of ER components, resulting in dysregulation of lipid metabolism and the accumulation of lipids within affected cells. It follows that by blocking or minimizing ER stress, it may be possible to attenuate the induction of lipid biosynthesis. In support of this concept, stable overexpression of GRP78/BiP, which protects cells from agents or conditions known to cause ER stress (Liu, H., et al 1998; and Morris, J. A., et al 1997), was observed to inhibit homocysteine-induced cholesterol gene expression in cultured human cells.

Under normal circumstances, SREBP activation is regulated by the SREBP cleavage activation protein (SCAP) according to the sterol requirements of the cell (Nohturfft, A., et al, 2000, Sakai J et al. 1996). However, the ER stress-driven activation of SREBP-1 and -2 observed in cells exposed to homocysteine appears to circumvent this control mechanism and thereby retain the cell in the “sterol starved” state despite lipid accumulation. As a result, endogenous lipid biosynthesis is maintained as is LDL receptor-mediated lipid uptake from plasma-derived lipoproteins a phenotype observed in HepG2 and HASMC treated with homocysteine. A similar response, involving ER stress, SREBP activation, elevated LDL receptor expression and marked cholesterol and triglyceride accumulation, occurs in the livers of mice with diet-induced hyperhomocysteinemia.

The ER-stress driven activation of SREBP may occur through dysregulation of the cellular machinery that normally controls SREBP function. For example, ER stress may moderate or abrogate the requirement of SCAP for SREBP translocation/activation. Alternatively, conditions of ER stress may activate SREBP via a separate cellular mechanism. In fact, ER stress has been shown to induce the proteolytic cleavage of another ER membrane bound transcription factor, ATF6 (Haze, K, et al. 1999, Wang, Y., et al. 2000).

Based upon the findings described herein, a mechanism is provided by which cells that are sensitive to elevated levels of homocysteine experience ER stress that leads to the activation and dysregulation the endogenous sterol response pathway. In mice with diet-induced hyperhomocysteinemia this results in localized lipid accumulation (i.e. hepatic steatosis), a condition observed in virtually all CBS-deficient patients having severe hyperhomocysteinemia. Such a homocysteine-induced cellular mechanism could also contribute to atherosclerotic lesion formation, especially in hyperhomocysteinemic individuals with normal serum lipid profiles.

TABLE 1
CBS (+/−) and wild type (CBS+/+) mice with diet-induced hyperhomocysteinemia
exhibit elevated levels of liver cholesterol and triglycerides.

| | | PLASMA | | | LIVER | |
|---|---|---|---|---|---|---|
| | | homocysteine^{B} | cholesterol | triglycerides | Cholesterol^{C} | triglycerides^{C} |
| Genotype | Diet^{A} | (μM) | (mM) | (mM) | (mg/mg protein) | (mg/mg protein) |
| CBS +/+ | control | 2.5 (0.9) | 0.91 (0.49) | 4.8 (0.8) | 0.018 (0.006) | 0.10 (0.02) |
| | HM | 8.8 (4.5) | 0.66 (0.30) | 5.7 (1.6) | 0.027 (0.002)* | 0.11 (0.01) |
| | HMLF | 60 (61) | 1.56 (0.40) | 6.7 (1.5) | 0.16 (0.04)* | 0.69 (0.31)* |
| CBS +/− | control | 6.2 (3.8) | 0.93 (0.45) | 6.0 (1.0) | 0.026 (0.003)† | 0.11 (0.03) |
| | HM | 27 (18) | 0.63 (0.27) | 5.5 (1.6) | 0.025 (0.001) | 0.12 (0.02) |
| | HMLF | 48 (63) | 1.41 (0.37) | 6.7 (1.5) | 0.33 (0.02)* | 0.39 (0.06)* |

^{A}Mice were fed control, high methionine (HM) or high methionine/low folate diets (HMLF) for 10 weeks.
^{B}All data are expressed as the means (±SD) (n = 4-8 mice/group).
^{C}Liver cholesterol and triglyceride concentrations are normalized to the total protein content of the tissue.
*P < 0.05: level of statistical significance (Student's t test) between the indicated values and the corresponding controls.
†P < 0.05: level of statistical significance (Student's t test) between CBS+/− and CBS+/+ controls.

### LIST OF REFERENCES

1. Ross, R. The pathogenesis of atherosclerosis: a perspective for the 1990s. 1993. Nature 362, 801-809.
2. McCully, K. S. Homocysteine and vascular disease. 1996. Nat. Med. 2, 386-389.
3. Ueland, P. M. and Refsum, H. 1989. Plasma homocysteine, a risk factor for vascular disease: plasma levels in health, disease, and drug therapy. J. Lab. Clin Med. 114, 473-501.
4. Clarke, R., Daly, L, Robinson, K., Naughten, E., Cahalane, S., Fowler, B. and Graham, I. 1991. Hyperhomocysteinemia: an independent risk factor for vascular disease. New Engl. J. Med. 324, 1149-1155.
5. Selhub, J, Jacques, P. F., Bostom, A. G., D'Agostino, R. B., Wilson, P. W., Belanger, A. J., O'Leary, D. H., Wolf, P. A., Schaefer, E. J. and Rosenberg, I. H. 1995. Association between plasma homocysteine concentrations and extracranial carotid-artery stenosis. New Engl. J. Med. 332, 286-291.
6. Welch, G. N. and Loscalzo, J. 1998. Homocysteine and atherothrombosis. New Engl. J. Med. 338, 1042-1050.
7. den Heijer, M., Koster, T, Blom, H. J., Bos, G. M., Briet, E., Reitsma, P. H., Vandenbroucke, J. P. and Rosendaal, F. R. 1996. Hyperhomocysteinemia as a risk factor for deep vein thrombosis. New Engl. J. Med. 334, 759-762.
8. Wilken, D. E. L. and Dudman, N. P. B. 1992. Homocystinuria and Atherosclerosis In: Molecular Genetics of Coronary Artery Disease, Lusis, A. J., Rotter, J. I. and Sparkes, R. S. (eds). Monogr. Hum. Genet. Basel, Karger, vol. 14, pp 311-324.
9. Harker, L. A., Slichter, S. J., Scott, C. R. and Ross, R. 1974. Homocystinemia: vascular injury and arterial thrombosis. N. Engl. J. Med. 291, 537-543.
10. Mudd, S. H., Levy, H. L. and Skovby, F. 1989. Disorders of transsulfation. In: The Metabolic Basis for Inherited Diseases. Scriver, C. R., Beadet, A. L., Sly, W. S. and Valle, D. eds. New York: McGraw-Hill, pp. 693-734.
11. Gaull, G, Sturman, J. A. and Schaffner, F. 1974. Homocystinuria due to cystathionine synthase deficiency: enzymatic and ultrastructural studies. J. Pediatr. 84, 381-390.
12. Watanabe, M, Osada, J., Aratani, Klucklman, K., Reddick, R. Malinow, M. R. and Maeda, N. 1995. Mice deficient in cystathionine β-synthase: animal models for mild and severe homocyst(e)inemia. Proc. Natl. Acad. Sci. USA 92, 1585-1589.
13. O. K., Lynn, E. G., Chung, Y. H., Siow, Y. L, Man, R. Y. and Choy, P. C. 1998. Homocysteine stimulates the production and secretion of cholesterol in hepatic cells. Biochimica et Biophyica Acta 1393, 317-324.
14. Zulli, A., Buxton, B., Doolan, L. and Liu, J. J. 1998. Effect of homocysteine and cholesterol in raising homocysteine, cholesterol and triglyceride levels. Life Sci. 62, 2192-2194.
15. Outinen, P. A., Sood, S. K., Pfeifer, S. I., Pamidi, S., Podor, T. J., Li J., Weitz, J. I. and Austin, R. C. 1999. Homocysteine-induced endoplasmic reticulum stress and growth arrest leads to specific changes in gene expression in human vascular endothelial cells. Blood 94, 959-967.
16. Outinen, P. A., Sood, S. K., Liaw, P. C. Y., Sarge, K. D., Maeda, N., Hirsh, J., Ribau, J., Podor, T. J., Weitz, J. I. and Austin, R. C. 1998. Characterization of the stress-inducing effects of homocysteine. Biochem. J. 332, 213-221.
17. Brown, M. S. and Goldstein, J. L. 1999. A proteolytic pathway that controls the cholesterol content of membranes, cells, and blood. Proc. Natl. Acad. Sci. USA 96, 11041-11048.
18. Horton, J. D. and Shimomura, I. 1999. Sterol regulatory element-binding proteins: activators of cholesterol and fatty acid synthesis. Curr. Opin. Lipidol. 10, 143-150.
19. Kokame, K., Kato, H. and Miyata, T. 1996. Homocysteine-respondent genes in vascular endothelial cells identified by differential display analysis: GRP78 and novel genes. J. Biol. Chem. 271, 29659-29665.
20. Miyata, T., Kokame, K., Agarwala, K. L. and Kato, H. 1998. Analysis of gene expression in homocysteine-injured vascular endothelial cells: demonstration of GRP78/BiP expression, cloning and characterization of a novel reducing agent-tunicamycin regulated gene. Sem. Thromb. Hemost. 24, 285-290.
21. Cai, Y, Zhang, C., Nawa, T, Aso, T. V. Tanaka, M., Oshiro, S., Ichijo, H. and Kitajima, S. 2000. Homocysteine-responsive ATF3 gene expression in human vascular endothelial cells: activation of c-Jun NH₂-terminal kinase and promoter response element. Blood 96, 2140-2148.
22. Wang, X. Z., Harding, H. P., Zhang, Y., Jolicoeur, E. M., Kuroda, M. and Ron, D. 1998. Cloning of mammalian Irel reveals diversity in the ER stress responses. EMBO J. 17, 5708-5717.
23. Cox, J. S., Chapman, R. E. and Walter, P. 1997. The unfolded protein response coordinates the production of endoplasmic reticulum protein and endoplasmic reticulum membrane. Mol. Biol. Cell 8, 1805-1814.
24. Doerrler, W. T. and Lehrman, M. A. 1999. Regulation of the dolichol pathway in human fibroblasts by the endoplasmic reticulum unfolded protein response. Proc. Natl. Acad. Sci. USA 96, 13050-13055.
25. Jaffe, E. A., Nachmann, R. L., Becker, C. G. and Minnick, C. R. 1973. Culture of human endothelial cells derived from umbilical veins: identification by morphologic and immunologic criteria. J. Clin. Invest. 52, 2745-2754.
26. Mudd, S. H., Finkelstein, J. D, Refsum, H., Ueland, P. M., Malinow, M. R., Lentz, S. R, Jacobsen, D. W., Brattstrom, L., Wilcken, B., Wilcken, D. E. et al. 2000. Homocysteine and its disulfide derivatives: a suggested consensus terminology. Arterioscler. Thromb. Vasc. Biol. 20, 1704-1706.
27. Lentz, S. R., Erger, R. A., Dayal, S., Maeda, N., Malinow, M. R., Heistad, D. D. and Faraci F. M. 2000. Folate dependence of hyperhomocysteinemia and vascular dysfunction in cystathionine □-synthase deficient mice. Am. J. Physol. Heart Circ. Physiol. 279, H970-H975.
28. Malinow, M. R., Sexton, G., Averbuch, M., Grossman, M., Wilson, D., and Upson, B. 1990. Homocyst(e)inemia in daily practice: levels in coronary artery disease. Coronary Artery Disease 1, 215-220.
29. Lentz, S. R., Malinow, M. P., Piegors, D. J., Bhopatkar-Teredesai, M., Faraci, F. M., and Heistad, D. D. 1997. Consequences of hyperhomocyst(e)inemia on vascular function in atherosclerotic monkeys. Arterioscler. Thromb. Vasc. Biol. 17, 2930-2934.
30. Brown, M. S., Faust, J. R., Goldstein, J. L., Kaneko, I. and Endo, A. 1978. Induction of 3-hydroxy-3-methylglutaryl coenzyme A reductase activity in human fibroblasts incubated with compactin (ML-236B), a competitive inhibitor of reductase. J. Biol. Chem. 253, 1121-1128.
31. Amemiya-Kudo, M., Shimano, H., Yoshikawa, T., Yahagi, N., Hasty, A. H., Okazaki, H., Tamura, Y., Yahagi, Y., Ohasi, K., Osuga, J. et al. 2000. Promoter analysis of the mouse sterol regulatory element-binding protein (SREBP)-1c gene. J. Biol. Chem. 275, 31078-31085.
32. Wang, X. Z., Lawson, B., Brewer, J. W., Zinszner, H., Sanjay, A., Mi, L. J., Boorstein, R. Kreibich, G. Hendershot, L. M. and Ron, D. 1996. Signals from the stressed endoplasmic reticulum induce C/EBP-homologous protein (CHOP/GADD153). Mol. Cell. Biol. 16, 4273-4280.
33. Horton, J. D., Shimano, H., Hamilton, R. L., Brown, M. S., and Goldstein, J. L. 1999. Disruption of LDL receptor gene in transgenic SREBP-1a mice unmasks hyperlipidemia resulting from production of lipid-rich VLDL. J. Clin Invest. 103, 1067-1076.
34. Selhub, J., Jacques, P. F., Wilson, P. W. F., Rush, D. and Rosenberg, I. H. 1993. Vitamin status and intake as primary determinants of homocysteinemia in an elderly population. J. Am. Med. Assoc. 270, 2693-2698.
35. Robinson, K., Mayer, E. L., Miller, D. P., Green, R., van Lente, F., Gupta, A., Kottke-Marchant, K., Savon, S. R., Selhub, J., Nissen, S. E. et al. 1995. Hyperhomocysteinemia and low pyridoxal phosphate: common and independent reversible risk factors for coronary artery disease. Circulation 92, 2825-2830.
36. Ubbink, I. B., van der Merwe, A., Delport, R, Allen, R. H., Stabler, S. P., Riezier, R. and Vermaak, W. I. 1996. The effect of a subnormal vitamin B-6 status on homocysteine metabolism. J. Clin. Invest. 98, 177-184.
37. Rolland, P. H., Friggi, A., Barlatier, A., Piquet, P., Latrille, V., Faye, M. M., Guillou, J3, Charpiot, P., Bodard, H, Ghiringhelli, O. et al. 1995. Hyperhomocysteinemia-induced vascular damage in the minipig. Circulation 91, 1161-1174.
38. Lentz, S. R., Sobey, C. G., Piegors, D. J., Bhopatkar, M. R. Faraci F. M., Malinow, M. R. and Heistad, D. D. 1996. Vascular dysfunction in monkeys with diet-induced hyperhomocyst(e)inemia. J. Clin. Invest. 98, 24-29.
39. Lentz, S. R., Malinow, M. R., Piegors, DJ., Bhopatkar-Teredesai, M., Faraci F. M. and Heistad, D. D. 1997. Consequences of hyperhomocyst(e)inemia on vascular function in atherosclerotic monkeys. Arterioscler. Thromb. Vasc. Biol. 17, 2930-2934.
40. Wall, R. T., Harlan, I. M., Harker, L. A. and Striker, G. E. 1980. Homocysteine-induced endothelial cell injury in vitro: a model for the study of vascular injury. Thromb. Res. 18, 113-121.
41. DeGroot, P. G., Willems, C, Boers, G. H. J., Gonsalves, M. D., VanAken, W. G. and Mourik, J. A. 1983. Endothelial cell dysfunction in homocystinuria Eur. J. Clin. Invest. 13, 405-410.
42. Starkebaum, G. and Harlan, J. M. 1986. Endothelial cell injury due to copper-catalyzed hydrogen peroxide generation from homocysteine. J. Clin. Invest. 77, 1370-1376.
43. Loscaizo, J. 19%. The oxidant stress of hyperhomocyst(e)inemia J. Clin Invest 98, 5-7.
44. Jabobsen, D. W. 2000. Hyperhomocysteinemia and oxidative stress: time for a reality check? Arterioscler. Thromb. Vasc. Biol. 20, 1182-1184.
45. Eberhardt, R. T., Forgione, M. A., Cap, A., Leopold, J. A., Rudd, M. A., Trolliet, M., Heydrick, S., Stark, R., Klings, E. S., Moldovan, N. I. et al. 2000. Endothelial dysfunction in a murine model of mild hyperhomocyst(e)inemia. J. Clin. Invest. 106, 483-491.
46. Fiskerstrand, T., Ueland, P. M. and Refsum, H. 1997. Folate depletion induced by methotrexate affects methionine synthase activity and its susceptibility to inactivation by nitrous oxide. J. Pharmacol. Exp. Ther. 282, 1305-1311.
47. Shimano, H., Horton, J. D., Hammer, R. E., Shimomura, I., Brown, M. S. and Goldstein, J. L. 1996. Overproduction of cholesterol and fatty acids causes massive liver enlargement in transgenic mice expressing truncated SREBP-1a. J. Clin. Invest. 98, 1575-1584.
48. Olszewski, A. J. and McCully, K. S. 1991. Homocysteine content of lipoproteins in hypercholesterolemia. Atherosclerosis 88, 61-68.
49. Li, L. J., Li, X., Ferrario, A., Rucker, N., Liu, E. S., Wong, S., Gomer, C. J. and Lee, A. S. 1992. Establishment of a Chinese hamster ovary cell line that expresses grp78 antisense transcripts and suppresses A23187 induction of both GRP78 and GRP94. J. Cell. Physiol. 153, 575-582.
50. Chapman, R., Sidrauski, C. and Walter, P. 1998. Intracellular signalling from the endoplasmic reticulum to the nucleus. Annu. Rev. Cell Dev. Biol. 14, 459-485.
51. Pahl, H. L. 1999. Signal transduction from the endoplasmic reticulum to the cell nucleus. Physiol. Rev. 79, 683-701.
52. Haze, K, Yoshida, H., Yanagi, H., Yura, T. and Mori, K. 1999. Mammalian transcription factor ATF6 is synthesized as a transmembrane protein ans activated by proteolysis in response to endoplasmic reticulum stress. Mol. Biol. Cell 10, 3787-3799.
53. Liu, H., Miller, E., van de Water, B. and Stevens, J. L. 1998. Endoplasmic reticulum stress proteins block oxidant-induced Ca²⁺ increases and cell death. J. Biol. Chem. 273, 12858-12862.
54. Morris, J. A., Dorner, A. J., Edwards, C. A., Hendershot, L. M. and Kaufman, R. J. 1997. Immunoglobulin binding protein (BiP) function is required to protect cells from endoplasmic reticulum stress but is not required for the secretion of selective proteins. J. Biol. Chem. 272, 4327-4334.
55. Nohturfft, A., Yabe, D, Goldstein, I. L., Brown, M. S. and Espenshade, P. J. 2000. Regulated step in cholesterol feedback localized to budding SCAP from ER membranes. Cell. 102, 315-323.
56. Sakai, J., Duncan, E. A., Rawson, R. B., Hua, X., Brown, M. S. and Goldstein, J. L. 1996. Sterol-regulated release of SREBP-2 from cell membranes requires two sequential cleavages, one within a transmembrane segment. Cell 85,1037-1046.
57. Wang, Y., Shen, J., Arenzana, N., Tussophon, W., Kaufman, R. J. and Prywes, R 2000. Activation of ATF6 and a ATF6 DNA binding site by the endoplasmic reticulum stress response.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications and patent documents cited in this application are incorporated by reference in their entirety for all purposes to the same extent as if each individual publication or patent document were so individually denoted.

1. A method of modulating cholesterol/triglyceride accumulation in a cell of a mammal, the method comprising:
modifying an ER stress response or ER stress in the cell by inducing expression of GRP78/BiP, and
reducing cholesterol/triglyceride accumulation in the cell of the mammal.

2. A method as claimed in claim 1 wherein the severity of, or the duration of the ER stress or ER stress response in the cell is reduced.

3. A method as claimed in claim 2 wherein the severity of, or the duration of the ER stress or ER stress response in the cell is reduced by (a) increasing the amount of, or inducing the activity or expression of GRP78/BiP.

4. (canceled)

5. A method of inhibiting the accumulation of cholesterol in a cell of a mammal, the method comprising
inhibiting an ER stress response in said cell by inducing expression of GRP78/BiP, and
inhibiting the accumulation of cholesterol in the cell of the mammal.

6. A method as claimed in claim 5 wherein the ER stress response is inhibited by (a) increasing the amount of, or inducing the activity or expression GRP78/BiP.

7. A method as claimed in claim 5, wherein said ER stress response is induced by homocysteine.

8. A method as claimed in claim 5, wherein said mammal has hyperhomocysteinemia.

9. A method as claimed in claim 5, wherein said ER stress response is induced by a viral infection.

10. A method as claimed in claim 5, wherein said ER stress response is induced by hypoxia.

11. A method as claimed in claim 5, wherein said accumulation of cholesterol is a result of an increased level of cholesterol biosynthesis in said cell.

12. A method as claimed in claim 5, wherein said accumulation of cholesterol is a result of an increased level of cholesterol uptake into said cell.

13. A method as claimed in claim 5, wherein said cell is an endothelial cell.

14. A method as claimed in claim 5, wherein said cell is a smooth muscle cell.

15. A method as claimed in claim 5, wherein said cell is a macrophage.

16. A method as claimed in claim 5, wherein said cell is a hepatic cell.

17. A method as claimed in claim 5, wherein said cell is present at an atherosclerotic lesion within said mammal.

18.-21. (canceled)

22. A method of inhibiting a cholesterol-associated disease or condition in a mammal, the method comprising:
inhibiting an ER stress response within a population of cells of said mammal, whereby the accumulation of cholesterol in said population of cells is inhibited by inducing expression of GRP78/BiP, and
inhibiting the cholesterol-associated disease or condition in the mammal.

23. A method as claimed in claim 22 wherein said accumulation of cholesterol is inhibited by inhibiting the level of cholesterol biosynthesis in said population of cells.

24. A method as claimed in claim 22 wherein said accumulation of cholesterol is inhibited by inhibiting the level of cholesterol uptake into said population of cells.

25. A method as claimed in claim 22 wherein the cholesterol-associated disease is atherosclerosis.

26. A method as claimed in claim 25 wherein said atherosclerosis in said mammal is induced by homocysteine.

27. A method as claimed in claim 26 wherein said mammal has hyperhomocysteinemia.

28. A method as claimed in claim 22, wherein said population of cells comprises endothelial cells.

29. A method as claimed in claim 22, wherein said population of cells comprises smooth muscle cells.

30. A method as claimed in claim 22, wherein said population of cells comprises macrophages.

31. A method as claimed in claim 22 wherein said population of cells comprises hepatic cells.

32. A method as claimed in claim 22 wherein said population of cells is present at an atherosclerotic lesion within said mammal.

33.-49. (canceled)
